SEQUENCE LISTING (22 sequences)

SEQ ID NO: 1 - DNA, 663 bp, Arabidopsis thaliana
atggagggccctttgttgagcgagtgtgaacacatcattgacattacaagcgatggtagttcttcatcaattgatggtcagcaaccacacgagggtgttgaattgggtgatccatacacaaatgaaagcatttgtaattcaatcgagtttgttgtgactttggtcttgattgctgcggtcataatcgttctgattgagccaagagatgaagaacatccacaaacaatgttgtctatatggatcatcggttatacatgtggctgtattgccactctccctatcctatgttggcgtttttggctttataaccgaagcgttggctccgaatctacagatgaatacttaagagagacaagaatcaacaaggatatggacttttttatgacgggtttcttcgtgggttggtatgtggtgtttttatggattctttcttccaaagcattcgactcatcagctctagatgatactacaactcaatacttctggttatgtttggctctccttactttcagttgcattagatatgttctttttaatctaacattggcaatggtctgttatactactcctttgcttttacttacgcctgtggtcatatgtgttctccttgctgtggaagtcttaaaaggcattgtagcatgcattgtatcatgcatctgctga

SEQ ID NO: 2 - DNA, 540 bp, Arabidopsis thaliana
atggacagcgaatcccacagtgataatgatgatcacataatcgacattacaaacgatgaagattcttcatcaagatcgtctttggacgagagaagctatccttcattaagttcgtctctgtctacggatgacgaatcgtcagatgaaggtgcaagttcatctacgagagactgtggtagtctttggaatataatggaattagtagtcactttggttcagattgttgcatcattaatagttttaactctggcaaaagatgaacatcaacaagcacttttgttaacatgggtcatcggttacacttgtggctgtattaccattacgctccttattctcctctcgtgtgtccgcaaatacaaccgaattggtgtttactcaaaaacgagaacggatagggtaatggatgctttgaagatggggaatgaatgtttctttgttgtgtggttagttatggggattctatggatttgttatggacactcatcttcttctgatactcctaagctctacagattctgtaagggaaggtataggaggtag

SEQ ID NO: 3 - DNA, 732 bp, Arabidopsis thaliana
atggagggccctttgttaaccaagagtgagcacaacgtcgaagacgttacaatccatggcgattcttcctcaaatgatgagcacatcatcgacattacaatcaatggcggttcgtcatcagctgatcatgagcaaactccacatgaggcatttcaatggagttgtccatttacaagtggaagtttctggattttggtcgagttagctgtgactttggtccagattgttgcagccatattttttctgattctaacaaaagatgaacgagatccagaactagatgtagctttgtttacatggatcatcttttatacttgtgcctctattgccactttgccttttgtatgttgccgtttatggcagtatatccaaaacgctagctcagagacaagcaacgaggtgatggagtaccgtttggaaaattttcttgaatctttcttcgtgtccttgattgtggtgtctttatggcatattctaaccgagtcatcatcacgtcaagatactagtcgacacttctggttatggctgggtctacttgctttcagttgcattcgatatgtgcttcctaatttccaatttaccgtacagttattcatgttgcctggggtgtcccttgttactgcacttgtggccgccatagtaacgatgattaattcgttactctcactctttttggtcatcattggactctttgcgttaaccttttcgtggatcattggaatgtgcggataa

SEQ ID NO: 4 - DNA, 738 bp, Arabidopsis thaliana
atggaggaccctttgttgactcagagtgagcacatcgtcgatgacgttacaatccatggcgattcttcttcaaatgaagagcacatcgtcgacgttacaaccaatggcaatccttcatcagctgatgagaaaagaccgcatgagggtgtccaatggagtgatatatttacatttaccactgtttgtattttggtcgagtttgttgtggctttagtccagattgttgccgccattgttgttctgactctggcaaaagatgaacaacctccacaaaaaatgtttcctacactgatcctcagttataccggttgctgtattgccacactccctattctaggtttgcgtttctggcattcttaccgaagtgttagcacagagacaagaatctacgaggtggtggacattttgaaaaagatgcttgaatatttcttcgtgggttgggttgtggtgcttttatggcatcttatcaacaactcatcatctatagataatactacgcaacagttctggttatgtatgactttccttgctatcagctgcattctacatgttcttcgtaatctcccctgtgcgggagtttgttttctgtatcctatgatactatatctttcccaatcgatagacttcgttggtgacattactgatgagataaatttgactacgtctataatcctattatgctttggaatctttgcctgcattatctgtggttgttgttccagatgcttatgcagataa

SEQ ID NO: 5 - DNA, 543 bp, Arabidopsis thaliana
atggagagccctttgttgagcaacgatgatgagcacatcgtcgacatcgacatgacagccgatgactatgcttcattaatttatacgctagtatcaattccaattgcaattgctttgaactcgattgagtttgttatgaccttggtccagattgtcgtagccatactagttgtgaaaggaacaaaaggtgaatatccagaagcaacatggatcctcgtctatgcttatggtagtattgccaatctccctattctttgttggcgtttctggcagatccgtcatcctgtctcacacttaagaatcaaccgagtcatgtttcgtttgaggacgatatttgaatgtttctttgcgggttggttcgtggtgttcttctgggtctttgtttgcagctcatcatctctagaccattctagtcaactcttctggttatgtgtggttttccttgttttcggttgcattcgatatgtgctccctatggtaatatgtgcagcgacgtgttgctgtgtgtttatgacgttatgttttaacgccgttaacacttga

SEQ ID NO: 6 - DNA, 789 bp, Arabidopsis thaliana
atggagagccctttgttgaccaagagtgagcacaacgtcgaagacgttacagtccatggcgatccttcctcaaatgaagagcacatagtcgacattacaaccaatggagattcttcaccagctgatgagcacatcgtggacattacaatcaatggcgattcgtcatcagctgaagagcaaactccacatgaggcttctcaatggagttgttcatttataaacggaattgtctggattttggtcgagttagctgtgactttgtcacagattgttgcagccacattttttctgactctgacaaaagatgaacagcatccagaactaaatcatgtacctttgttaatatggatcatctgctatacttgtgcctctattgccactgtacctattgtatgttgccgtttatggcaatatatccgaaccgctagatcagggacatgttacgagttgatcggtttggaaaattttcttgaaacttgcttcgtctgtttggttgtgatgtttttgttgggttttttaacggagttatcatcacgtgatcctagtactagtcaacacttctggttatggctggctctaattgctttcagttgcattcgatatctgcttcctaatctcacatgtgtcaaagagtgcttcgtgtggcctgtgcttttccttgttaaacaactttgggaaggcataatagcaatgattgatgcgttaactgcactcattgggctcatcattgcaatcattgggtacatcttatttttgatcctttcaatctttatgtacatctgcggataa

SEQ ID NO: 7 - DNA, 768 bp, Arabidopsis thaliana
atggaggggcctttgttgactaagaatgagcacatcctcgacgacgttacaataatccatggcgattcttcctcaaacgaagagcacatcgtcaacattacaaccaatgtcgattcttcatcagatgatgagcaaacacaggatgattgtggtgatcatttgagacgtataatagctctttttgtggatgaagggtgtgatggatttacatctagtcatgtttgggttatggtcgagtttgttttgactttgctccaggttgttgcagccattgctgtcctgactctgacaaaagatgaaacagatccacagaaagtgtttcgtacactgatcatctgttataccggtggctgtattgtcgtgctccttattctaggtttggatttctgggattattgcggaagattctacgaggtgatggagaatttgaaaaagatgcttgactatttcttcgtgggttgggttgtggtgttttcatggcatcttataaacaactcatcatctccagataatgctacacaacagtactggttatgtatggctttccttgttgtcagttgcattctacatgtgcttcctaatctcccctgcgcggcggcttgttttctatatcctatgatacttcgtcttacccagtctatagacttctttgatgacattactgaaaagatagaggacattaactggttaatctttgtatacttttttggaatcttctcctgcattatctgttgcatttttacttgttgcagcagattatgtagataa

SEQ ID NO: 8 - DNA, 684 bp, Arabidopsis thaliana
atggaggggcctttgctaaccaagaacaagcacatcaaaatccatggcgattcttcatcaaatgatgagcataatgtcaacattacaatcactaacgattcttcatcaacaccacacggcgttaatattgttgaacggtgtattccatgcacaagagaaatggagtggttttgtttcatcgagtttgttttgaatattgtccaaattgttgcagcttttgtagttgtgactcgggcaaaagatgaacatccaggaacatcatttcttgtatggatcattggttacacttgtggatgtgttgccattcttcttattcagtttattaaccgtatttcgtcaagaagctacgaggagatcatggacgttttgaagaatatatgtgaatatttcttcgttggttgggttgtgttgtttctttggatgtatcatagtagttcttcatctctatatgataatactcaatatttctggttatgtatggcttttcttgctttcacttgcattcgatatgtaccggctcgtttaatatgttcagcaatatgctttattttcgttgtgataatatggttttgcgccgatgtgttaggcacaaccccgagtatgtttgctaatgctgtattccttatagttttcattggaattcttaaagtcattgaggaggttattggatcctattgttgttga

SEQ ID NO: 9 - protein, 220 aa, Arabidopsis thaliana
MEGPLLSECEHIIDITSDGSSSSIDGQQPHEGVELGDPYTNESICNSIEFVVTLVLIAAVIIVLIEPRDEEHPQTMLSIWIIGYTCGCIATLPILCWRFWLYNRSVGSESTDEYLRETRINKDMDFFMTGFFVGWYVVFLWILSSKAFDSSALDDTTTQYFWLCLALLTFSCIRYVLFNLTLAMVCYTTPLLLLTPVVICVLLAVEVLKGIVACIVSCIC

SEQ ID NO: 10 - protein, 179 aa, Arabidopsis thaliana
MDSESHSDNDDHIIDITNDEDSSSRSSLDERSYPSLSSSLSTDDESSDEGASSSTRDCGSLWNIMELVVTLVQIVASLIVLTLAKDEHQQALLLTWVIGYTCGCITITLLILLSCVRKYNRIGVYSKTRTDRVMDALKMGNECFFVVWLVMGILWICYGHSSSSDTPKLYRFCKGRYRR

SEQ ID NO: 11 - protein, 234 aa, Arabidopsis thaliana
MEGPLLTKSEHNVEDVTIHGDSSSNDEHIIDITINGGSSSADHEQTPHEAFQWSCPFTSGSFWILVELAVTLVQIVAAIFFLILTKDERDPELDVALFTWIIFYTCASIATLPFVCCRLWQYIQNASSETSNEVMEYRLENFLESFFVSLIVVSLWHILTESSSRQDTSRHFCCIRYVLPNFQFTVQLFMLPGVSLVTALVAAIVTMINSLLSLFLVIIGLFALTFSWIIGMCG

SEQ ID NO: 12 - protein, 245 aa, Arabidopsis thaliana
MEDPLLTQSEHIVDDVTIHGDSSSNEEHIVDVTTNGNPSSADEKRPHEGVQWSDIFTFTTVCILVEFVVALVQIVAAIVVLTLAKDEQPPQKMFPTLILSYTGCCIATLPILGLRFWHSYRSVSTETRIYEVVDILKKMLEYFFVGWVVVLLWHLINNSSSIDNTTQQFWLCMTFLAISCILHVLRNLPCAGVCFLYPMILYLSQSIDFVGDITDEINLTTSIILLCFGIFACIICGCCSRCLCR

SEQ ID NO: 13 - protein, 180 aa, Arabidopsis thaliana
MESPLLSNDDEHIVDIDMTADDYASLIYTLVSIPIAIALNSIEFVMTLVQIVVAILVVKGTKGEYPEATWILVYAYGSIANLPILCWRFWQIRHPVSHLRINRVMFRLRTIFECFFAGWFVVFFWVFVCSSSSLDHSSQLFWLCVVFLVFGCIRYVLPMVICAATCCCVFMTLCFNAVNT

SEQ ID NO: 14 - protein, 262 aa, Arabidopsis thaliana
MESPLLTKSEHNVEDVTVHGDPSSNEEHIVDITTNGDSSPADEHIVDITINGDSSSAEEQTPHEASQWSCSFINGIVWILVELAVTLSQIVAATFFLTLTKDEQHPELNHVPLLIWIICYTCASIATVPIVCCRLWQYIRTARSGTCYELIGLENFLETCFVCLVVMFLLGFLTELSSRDPSTSQHFWLWLALIAFSCIRYLLPNLTCVKECFVWPVLFLVKQLWEGIIAMIDALTALIGLIIAIIGYILFLILSIFMYICG

SEQ ID NO: 15 - protein, 255 aa, Arabidopsis thaliana
MEGPLLTKNEHILDDVTIIHGDSSSNEEHIVNITTNVDSSSDDEQTQDDCGDHLRRIIALFVDEGCDGFTSSHVWVMVEFVLTLLQVVAAIAVLTLTKDETDPQKVFRTLIICYTGGCIVVLLILGLDFWDYCGRFYEVMENLKKMLDYFFVGWVVVFSWHLINNSSSPDNATQQYWLCMAFLVVSCILHVLPNLPCAAACFLYPMILRLTQSIDFFDDITEKIEDINWLIFVYFFGIFSCIICCIFTCCSRLCR

SEQ ID NO: 16 - protein, 227 aa, Arabidopsis thaliana
MEGPLLTKNKHIKIHGDSSSNDEHNVNITITNDSSSTPHGVNIVERCIPCTREMEWFCFIEFVLNIVQIVAAFVVVTRAKDEHPGTSFLVWIIGYTCGCVAILLIQFINRISSRSYEEIMDVLKNICEYFFVGWVVLFLWMYHSSSSSLYDNTQYFWLCMAFLAFTCIRYVPARLICSAICFIFVVIIWFCADVLGTTPSMFANAVFLIVFIGILKVIEEVIGSYCC

SEQ ID NO: 17 - DNA, 2525 bp, Arabidopsis thaliana
tccgggaagacctagccgtagttcggtgatttcgagtccaagattattactctcattcgccatctttgatgtttttgattgaaagtattggtgttaatgtgtttgaactttattctgttgccactttatatgttctcatgggaacataaacgatggcgtttgagacaagcttgtttagcattgatttatcttgtcggccccattcgaagatcttgtcgaaatttttatgtcatgttttttcattcctttgccgcttcgtgtttaaatgtcgataatatggatccgttggttcgacacactggacggccacgattttgttaccgacaaagtagggtttctttctcagccgttggatcagaaacacgttactttctattagacatcccatgttatgtgtgatttgttatgtctgtatcccattcttttaatcaacgacataataataaaacaacatattcatggtcgaatatatattagtgcactttaaaatctacgtatatttatgtttctcgacaaccgaacacgtgttactcaaggctaagacaagttattatattatttcatttttttctacacgtaacacactacacagtgcatacataatttcaaagaaaaaaatgcatctacgttttcgtcatatatcttgtttctgctattcacacatgtgcccgatactttcattacatcatacgtaatctacacatgtacaccatacttttgtcattatttgttacgaaatagaaacagaactgatcaaaatatcaattacgggatatatttccatttttcatagatatttccatatctttgtatctccaatagtacatttttggaatgttcacatgttgatagataagctagattagtcaaatttccttgtacacaatttctagttttcgattgatcatgtagaattgcgtataatatattgtcggctggctttagccaatgaataacacaacaagactttgtgataatggacagcgaatcccacagtgataatgatgatcacataatcgacattacaaacgatgaagattcttcatcaagatcgtctttggacgagagaagctatccttcattaagttcgtctctgtctacggatgacgaatcgtcagatgaaggtgcaagttcatctacgagagactgtggtagtctttggaatataatggaattagtagtcactttggttcagattgttgcatcattaatagttttaactctggcaaaagatgaacatcaacaagcacttttgttaacatgggtcatcggttacacttgtggctgtattaccattacgctccttattctcctctcgtgtgtccgcaaatacaaccgaattggtgtttactcaaaaacgaggtataataaacaacacttgtttcacttaacattaagagtttaattagctaaatcaaaccgaaccgttgttggtggcagaacggatagggtaatggatgctttgaagatggggaatgaatgtttctttgttgtgtggttagttatggggattctatggatttgttatggacactcatcttcttctgatactcctaagctctacaggtttgactcacaatcatgtcacagtagagatttagttaaaccctaactaatcagtactaacttttgtcttcttgattatgttttggcaggttatgtgtagtcttcattgctttcagttgcattagatttgcatatgcattactattatgcgcaggtatttttttcgcttaatgttcactagagtttggaacatttttttctcatatctatgatgtggataagttgaaatcaaatgatgtagattctgtaagggaaggtataggaggtagatttgttttcaagaaaccgagccatgatgatgtgagtaataatttcatatcacattaattactctgttttaaatagcatttttttgttgttgttgttgtttaattttcatggtttgatggtgtgtgcagagttgttgcatatgcttggggaaatatggagaggaaaaaggagtaggactaaggaaacttgaatgctcacacgtatttcattcgcaatgtatagacaaatggctgaaaatcaaatccacatgccctctctgtcaatcacaagttaggtggtgatgagatgaggttgattttttttttatttttttttcgctcaagagtaattgtacacattcaataaacaaaagtggtgggaaactgaaagatacgatcctgtcttcttgtgttgtctgtcttaaaacagaaaataaatatcatacagatcatttgacctattaacaatttaacttgtccaaaacatctgcaatatatataaacattgaaatctcagctttgatcttcatttttcaaaataatgttttatttgtaacacgactaatacgtacttagtattgatctggtctaaaaaaatactaataattttatcaacaaagaaggaaaggtatatgctttgtgctcgtcaacgcaaatttgaatgaacttttcatttgatcggcaagaaataggaaaaaccaaaagtgatca

SEQ ID NO: 18 - DNA, 20 bp, Artificial Sequence
  DNA encoding gRNA
gatggtcagcaaccacacga

SEQ ID NO: 19 - DNA, 24 bp, Artificial Sequence
  primer
acttctggttatgtttggctctcc

SEQ ID NO: 20 - DNA, 23 bp, Artificial Sequence
  primer
acacatatgaccacaggcgtaag

SEQ ID NO: 21 - DNA, 24 bp, Artificial Sequence
  primer
attgcgtattgccactctcatagg

SEQ ID NO: 22 - DNA, 22 bp, Artificial Sequence
  primer
tcctgacagggataccgaatgc

(19) United States
(12) Patent Application Publication
(10) Pub. No.: US 2021/0269815 A1
(43) Pub. Date: Sep. 2, 2021

(54) GENETICALLY MODIFIED PLANT AND USES THEREOF

(71) Applicant: IST AUSTRIA, Klostemeuburg (AT)

(72) Inventors: Eva BENKOVA, Klosterneuburg (AT); Andrej HURNY, Klosterneuburg (AT); Nicola CAVALLARI, Klosterneuburg (AT)

(73) Assignee: IST AUSTRIA, Klosterneuburg (AT)

(21) Appl. No.: 17/255,501

(22) PCT Filed: Jun. 28, 2019
(86) PCT No.: PCT/EP2019/067451

§ 371 Date: Dec. 23, 2020

(30) Foreign Application Priority Data
Jun. 28, 2018 (GB) 1810631.0

(51) Int. Cl. C12N 15/82

(52) U.S. Cl. CPC C12N 15/8246; C12N 15/8294; C12N 15/8271; C12N 15/8279; C12N 15/8295

(57) ABSTRACT

The invention provides a genetically modified plant or plant cell transformed with an isolated nucleic acid, wherein the isolated nucleic acid encodes a SYAC1 protein or a protein related to the SYAC1 protein or a protein with at least about 80%, 85%, 90%, 95%, 98%, 99% or more sequence identity protein with a SYAC1 protein. The invention also provides a genetically modified plant or plant cell engineered to express a reduced level, or no, SYAC1 protein. A genetically modified plant according to the invention may be more resistant to infection by Plasmodiophora brassicae. Alternatively, the invention provides a genetically modified plant with increased levels of SYAC1 protein or related protein which is more receptive to beneficial microorganisms and/or grows more effectively on land contaminated with heavy metals.

The present invention relates to genetically modified plants, and in particular to genetically modified plants which comprise the stable and/or transient expression of SYAC1 (synergistic on auxin and cytokinin 1) or a related gene. Preferably the expression or increased expression of the SYAC1 gene or related gene, or the expression or increased expression of the SYAC1 protein or related protein, results in an increase of and/or easier access to the sugars in the plant cell wall. The expression or increased expression of the SYAC1 gene or related gene, or the expression or increased expression of the SYAC1 protein or related protein, may also, or alternatively, reduce intake of metal particles/ions on contaminated soil or allow beneficial microorganism to more readily interact with the plant. Alternatively, the genetically modified plants may be modified to reduce or eliminate expression of the SYAC1 gene or related gene, or to reduce or eliminate expression of the SYAC1 protein or related protein. Such plants may display improved resistance to one or more plant pathogen.

Currently, fossil fuels (petroleum, coal, and natural gas) are the major primary energy sources. However, fossil fuels face a number of problems, including a fast depletion, environmental damage, and global warming. It is widely accepted that the existing fossil fuels should be replaced in future by renewable energy sources such as biomass, solar, wind, and geothermal. At present biomass is the fourth largest energy resource in the world, after petroleum, gas and coal. The key features of biomass include renewability and neutral CO₂ impact.

Biomass can be converted into all major energy carriers such as electricity, heat, and transport fuels as well as a wide diversity of chemicals and materials that are currently produced from fossil fuels. An alternative source of transport fuel is biofuel, and in particular biofuel produced from plant biomass. The market for biofuels is rapidly growing. From a market size of around $9 billion in 2016, it is predicted to grow to about $100 billion by 2023 (https://globenewswire.com/news-release/2018/01/09/1285912/0/en/Biofuels-Market-Size-Will-Reach-USD-218-7-Billion-by-2022-Globally-Zion-Market-Research.html).

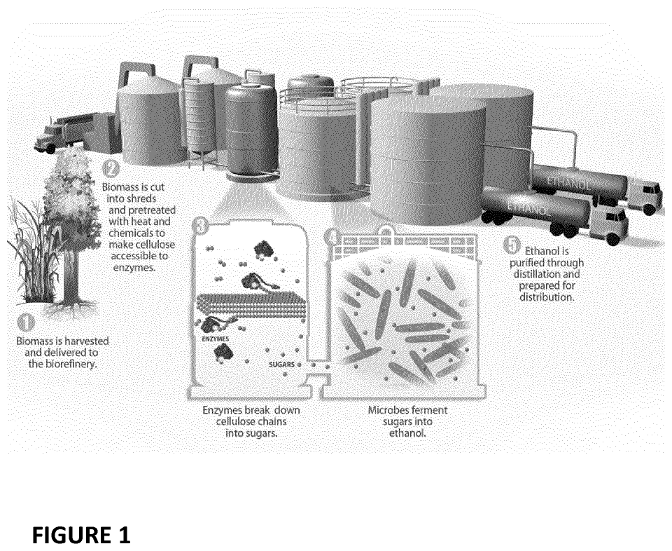

On the other hand, the use of biomass is accompanied by several drawbacks, mainly limitations of land and water, and competition with food production. Approximately 70% of plant biomass is estimated to be present in plant cell walls. Current practices use only about 2% of plant cell wall-based biomass, there is therefore a great opportunity to use this valuable resource as a raw material for the production of biofuels and commodity chemicals. The plant cell wall provides mechanical support to the plant and contributes to plant growth and development. Carbohydrates, proteins and phenolics (e.g., lignin) compounds are the major components in the plant cell wall together with cellulose, hemicellulose and pectin comprising the major polysaccharides in the wall. The goal of using bioenergy crops for bio-ethanol production is well established. However, cost effectiveness is one of the major limitations for this industry and is intimately associated with biomass recalcitrance. A major barrier to the use of biomass as an energy source is the cost of the bacterial and fungal enzymes needed to degrade the plant cell wall. Therefore, there is a need to produce plants with genetically modified cell walls from which sugars can more easily be released. Such plants would serve as raw materials for the bio-ethanol industry. The typical process to produce biofuels from plants is illustrated in FIG. 1.

An aim of the present invention is to modify plant material used as the biomass in biofuel production such that sugars embedded in plant cell walls are more readily available to the enzymes used in biofuel production and thus the process is more efficient and cost effective. In this invention this may be obtained by one or both of 1) increasing the sugar content of the plant cell walls; and 2) decreasing the resistance of the cell walls to degradation.

Cotton is one of the world's most commonly used natural fibers. It is composed of almost pure cellulose and its production reached approximately 25 million tons annually in 2011. Despite its large market cotton, has serious impacts on people and environment. Cotton cultivation extensively uses herbicides, fertilizers and insecticides and even though only 2.5% of the world's cultivated land is cotton, it accounts for 16% of the world's pesticide use. Although some cotton is produced according to strict consideration for the environment the majority of the world production is produced using intensive techniques. Cotton production has high maintenance and energy costs and frequently uses extreme quantities of water, leaving soil depleted of nutrients, reducing soil fertility and biodiversity, and favoring soil salinization.

As water resources get tighter around the world, cotton cultivation is creating economic difficulties in several countries that rely on it, as well as causing potential environmental problems. Improper cotton cropping and irrigation practices have led to desertification in areas of Uzbekistan, where cotton is a major export. In the days of the Soviet Union, the Aral Sea was tapped for agricultural irrigation, largely for cotton production, and now salinization is widespread.

Other fibers are being considered as alternatives to cotton, for example, a large number of natural fibers can be extracted from the stem (Jute, flax, linen, ramie and hemp; bast fibers), leaves (Agave and yucca), seeds (Coconut) and other plant parts (Bamboo). Research and development is starting to allow the processing and the use of these fibers to create new economically viable cultivation. Some of these plants, like hemp, can be grown with almost no use of pesticides, on smaller areas of land and can be used to create a durable, sustainable and renewable fiber source.

However, the extraction and cleaning of fibers from plant material is expensive, principally because the process involves several steps like scraping, pounding, heating, washing, or exposure to chemicals in order to separate the raw fiber from the gums or resins in which it is embedded in the plant tissue.

Another aim of the present invention is to modify plant material used in the extraction of natural fibers in order to make the process more efficient and cost effective. In this invention this may be obtained by one or both of 1) increasing the sugar content of the plant cell walls (cellulose and other polysaccharide); and 2) decreasing the protein composition of the cell wall increasing the quantity and the quality of the fiber content.

The invention may also provide plants suitable for cultivation on contaminated soils. In particular, on soil contaminated with heavy metals, such as copper and/or iron. It is reported that as a result of industrial activity arable soils all round the world are becoming polluted with heavy metals, the most urgent situation is in China, where more than 19% of the farmland soil is polluted. This pollution has a devastating effect on the productivity of the land, as well as the bio-accumulation of metals in plants which may then enter the food chain; the affected crops may have to be destroyed. There is therefore the need for plants that can be cultivated on contaminated soil which will produce crops that are not harmful for human or animal consumption. Plants of this invention are able to regulate/reduce the uptake of the contaminating heavy metals, and thus can provide an alternative or a parallel strategy to soil phytoremediation. Soil phytoremediation is a cost-effective approach aimed at reducing the quantity of toxic compounds from the environment which accumulate in plant tissues. However, plant growth and low biomass are limiting steps which imply a long term commitment for this technique. The modified plants in this invention may be cultivated on contaminated soils bypassing these limiting steps in canonical phytoremediation.

Another aim of the invention is to modify plants to provide protection against plant pathogens. In the heterogeneous soil environment plant roots are exposed to a large variety of biotic and abiotic factors Rhizospheric microbes are among the prominent biotic factors which have developed different strategies, including the modification of phytohormone responses, to penetrate, colonize and hijack nutrients from host plants. The present invention is particularly concerned with protecting plants from infection by Plasmodiophora brassicae, the causal agent of clubroot disease in cruciferous plants including Arabidopsis. Clubroot disease is one of the most damaging diseases threatening the agriculture and food sector. In the years 2017-2018 as much as 30 to 70% of surveyed fields were confirmed to have symptoms of this disease all over the world. Clubroot is caused by the obligate biotrophic protist Plasmodiophora brassicae and it is harmful for most profitable commodities such as canola, rapeseed, mustard, cabbage, broccoli, cauliflower and many others plants from the Brassicaceae family. Root growth of infected plants is disrupted and the roots become malformed due to increased cell division leading to development of characteristic galls. Water and nutrient uptake is restricted by the gall groups and this may result in reduced seed production, stunting and premature death of the plant. Currently, there are no economical control measures that can remove this pathogen from a field once it has become infested (chemical protection against it is complicated, environmentally unfriendly and impossible on large areas). However, by crop rotation and use of resistant crop variants it is possible to curtail the spread of the pathogen and reduce the incidence and severity of the disease.

Another aim of the invention is to provide a genetically modified plant comprising one or more cells which have been modified to be more susceptible to beneficial microorganisms. This may be achieved by engineering the levels of SYAC1 protein, or a related protein, in one or more cells to alter the cell wall composition and thus allow beneficial microorganism to more readily enter and to increase colonisation efficiency. One example would be to promote colonization by nitrogen-fixing bacteria.

In one aspect the present invention provides a genetically modified plant or plant cell transformed with an isolated nucleic acid, wherein the isolated nucleic acid encodes a SYAC1 protein or a protein related to the SYAC1 protein or a protein with at least about 80%, 85%, 90%, 95%, 98%, 99% or more sequence identity protein with a SYAC1 protein or a SYAC1 related protein. The isolated nucleic acid may comprise or consist of a sequence selected from the group consisting of Seq ID no: 1, Seq ID no: 2, Seq ID no: 3, Seq ID no: 4, Seq ID no: 5, Seq ID no: 6, Seq ID no: 7 or Seq ID no: 8, or a sequence that has at least 80%, 85%, 90%, 95%, 98%, 99% or more sequence identity with one of Seq ID no: 1, Seq ID no: 2, Seq ID no: 3, Seq ID no: 4, Seq ID no: 5, Seq ID no: 6, Seq ID no: 7 or Seq ID no: 8.

A related genet to SYAC1 may be a gene paralogous to SYAC1, the gene may be located in the same cluster on the chromosome as SYAC1.

A protein related to SYAC1 may include a protein encoded by a paralogous gene to SYAC1, these may be located in the same cluster on the chromosome as SYAC1.

In another aspect the present invention provides a genetically modified plant or plant cell transformed with an isolated nucleic acid, wherein the isolated nucleic acid encodes a polypeptide having the sequence of Seq ID no: 9, Seq ID no: 10, Seq ID no: 11, Seq ID no: 12, Seq ID no: 13, Seq ID no: 14, Seq ID no: 15 or Seq ID no: 16, or a sequence that has at least about 80%, 85%, 90%, 95%, 98%, 99% or more sequence identity with one of Seq ID no: 9, Seq ID no: 10, Seq ID no: 11, Seq ID no: 12, Seq ID no: 13, Seq ID no: 14, Seq ID no: 15 or Seq ID no: 16.

The percent identity of two amino acid sequences or of two nucleic acid sequences is generally determined by aligning the sequences for optimal comparison purposes (e.g., gaps can be introduced in the first sequence for best alignment with the second sequence) and comparing the amino acid residues or nucleotides at corresponding positions. The “best alignment” is an alignment of two sequences that results in the highest percent identity. The percent identity is determined by comparing the number of identical amino acid residues or nucleotides within the sequences (i.e., % identity=number of identical positions/total number of positions×100).

The determination of percent identity between two sequences can be accomplished using a mathematical algorithm known to those of skill in the art. An example of a mathematical algorithm for comparing two sequences is the algorithm of Karlin and Altschul (1990), modified as in Karlin and Altschul (1993). The NBLAST and XBLAST programs of Altschul et al. (1990) have incorporated such an algorithm. BLAST nucleotide searches can be performed with the NBLAST program, score=100, word length=12 to obtain nucleotide sequences homologous to a nucleic acid molecules of the invention. BLAST protein searches can be performed with the XBLAST program, score=50, word length=3 to obtain amino acid sequences homologous to a protein molecules of the invention. To obtain gapped alignments for comparison purposes, Gapped BLAST can be utilized as described in Altschul et al. (1997). Alternatively, PSI-Blast can be used to perform an iterated search that detects distant relationships between molecules. When utilizing BLAST, GappedBLAST, and PSI-Blast programs, the default parameters of the respective programs (e.g., XBLAST and NBLAST) can be used. See http://www.ncbi.nlm.nih.gov. Another example of a mathematical algorithm utilized for the comparison of sequences is the algorithm of Myers and Miller. The ALIGN program (version 2.0) which is part of the GCG sequence alignment software package has incorporated such an algorithm. Other algorithms for sequence analysis known in the art include ADVANCE and ADAM as described in Torellis and Robotti (1994); and FASTA described in Pearson and Lipman (1988). Within FASTA, ktup is a control option that sets the sensitivity and speed of the search.

The sequences may also include a small number of functionally inconsequential amino acid substitutions (e.g., conservative substitutions), deletions, or insertions.

AT1G15600 CDS (SYAC1)
Seq ID no 1 is:
ATGGAGGGCCCTTTGTTGAGCGAGTGTGAACACATCATTGACATTACAAGCGATGGTAGTTCT
TCATCAATTGATGGTCAGCAACCACACGAGGGTGTTGAATTGGGTGATCCATACACAAATGAA
AGCATTTGTAATTCAATCGAGTTTGTTGTGACTTTGGTCTTGATTGCTGCGGTCATAATCGTTCT
GATTGAGCCAAGAGATGAAGAACATCCACAAACAATGTTGTCTATATGGATCATCGGTTATAC
ATGTGGCTGTATTGCCACTCTCCCTATCCTATGTTGGCGTTTTTGGCTTTATAACCGAAGCGTTG
GCTCCGAATCTACAGATGAATACTTAAGAGAGACAAGAATCAACAAGGATATGGACTTTTTTA
TGACGGGTTTCTTCGTGGGTTGGTATGTGGTGTTTTTATGGATTCTTTCTTCCAAAGCATTCGA
CTCATCAGCTCTAGATGATACTACAACTCAATACTTCTGGTTATGTTTGGCTCTCCTTACTTTCA
GTTGCATTAGATATGTTCTTTTTAATCTAACATTGGCAATGGTCTGTTATACTACTCCTTTGCTTT
TACTTACGCCTGTGGTCATATGTGTTCTCCTTGCTGTGGAAGTCTTAAAAGGCATTGTAGCATG
CATTGTATCATGCATCTGCTGA
AT1G15590 CDS (Brother of SYAC1)
Seq ID no: 2 is:
ATGGACAGCGAATCCCACAGTGATAATGATGATCACATAATCGACATTACAAACGATGAAGAT
TCTTCATCAAGATCGTCTTTGGACGAGAGAAGCTATCCTTCATTAAGTTCGTCTCTGTCTACGG
ATGACGAATCGTCAGATGAAGGTGCAAGTTCATCTACGAGAGACTGTGGTAGTCTTTGGAATA
TAATGGAATTAGTAGTCACTTTGGTTCAGATTGTTGCATCATTAATAGTTTTAACTCTGGCAAA
AGATGAACATCAACAAGCACTTTTGTTAACATGGGTCATCGGTTACACTTGTGGCTGTATTACC
ATTACGCTCCTTATTCTCCTCTCGTGTGTCCGCAAATACAACCGAATTGGTGTTTACTCAAAAAC
GAGAACGGATAGGGTAATGGATGCTTTGAAGATGGGGAATGAATGTTTCTTTGTTGTGTGGT
TAGTTATGGGGATTCTATGGATTTGTTATGGACACTCATCTTCTTCTGATACTCCTAAGCTCTAC
AGATTCTGTAAGGGAAGGTATAGGAGGTAG
AT1G1510 CDS
Seq ID no 3 is:
ATGGAGGGCCCTTTGTTAACCAAGAGTGAGCACAACGTCGAAGACGTTACAATCCATGGCGA
TTCTTCCTCAAATGATGAGCACATCATCGACATTACAATCAATGGCGGTTCGTCATCAGCTGAT
CATGAGCAAACTCCACATGAGGCATTTCAATGGAGTTGTCCATTTACAAGTGGAAGTTTCTGG
ATTTTGGTCGAGTTAGCTGTGACTTTGGTCCAGATTGTTGCAGCCATATTTTTTCTGATTCTAAC
AAAAGATGAACGAGATCCAGAACTAGATGTAGCTTTGTTTACATGGATCATCTTTTATACTTGT
GCCTCTATTGCCACTTTGCCTTTTGTATGTTGCCGTTTATGGCAGTATATCCAAAACGCTAGCTC
AGAGACAAGCAACGAGGTGATGGAGTACCGTTTGGAAAATTTTCTTGAATCTTTCTTCGTGTC
CTTGATTGTGGTGTCTTTATGGCATATTCTAACCGAGTCATCATCACGTCAAGATACTAGTCGA
CACTTCTGGTTATGGCTGGGTCTACTTGCTTTCAGTTGCATTCGATATGTGCTTCCTAATTTCCA
ATTTACCGTACAGTTATTCATGTTGCCTGGGGTGTCCCTTGTTACTGCACTTGTGGCCGCCATA
GTAACGATGATTAATTCGTTACTCTCACTCTTTTTGGTCATCATTGGACTCTTTGCGTTAACCTTT
TCGTGGATCATTGGAATGTGCGGATAA
AT1G15620 CDS
Seq ID no 4 is:
ATGGAGGACCCTTTGTTGACTCAGAGTGAGCACATCGTCGATGACGTTACAATCCATGGCGAT
TCTTCTTCAAATGAAGAGCACATCGTCGACGTTACAACCAATGGCAATCCTTCATCAGCTGATG
AGAAAAGACCGCATGAGGGTGTCCAATGGAGTGATATATTTACATTTACCACTGTTTGTATTTT
GGTCGAGTTTGTTGTGGCTTTAGTCCAGATTGTTGCCGCCATTGTTGTTCTGACTCTGGCAAAA
GATGAACAACCTCCACAAAAAATGTTTCCTACACTGATCCTCAGTTATACCGGTTGCTGTATTG
CCACACTCCCTATTCTAGGTTTGCGTTTCTGGCATTCTTACCGAAGTGTTAGCACAGAGACAAG
AATCTACGAGGTGGTGGACATTTTGAAAAAGATGCTTGAATATTTCTTCGTGGGTTGGGTTGT
GGTGCTTTTATGGCATCTTATCAACAACTCATCATCTATAGATAATACTACGCAACAGTTCTGG
TTATGTATGACTTTCCTTGCTATCAGCTGCATTCTACATGTTCTTCGTAATCTCCCCTGTGCGGG
AGTTTGTTTTCTGTATCCTATGATACTATATCTTTCCCAATCGATAGACTTCGTTGGTGACATTA
CTGATGAGATAAATTTGACTACGTCTATAATCCTATTATGCTTTGGAATCTTTGCCTGCATTATC
TGTGGTTGTTGTTCCAGATGCTTATGCAGATAA
AT1G15625 CDS
Seq ID no 5 is:
ATGGAGAGCCCTTTGTTGAGCAACGATGATGAGCACATCGTCGACATCGACATGACAGCCGA
TGACTATGCTTCATTAATTTATACGCTAGTATCAATTCCAATTGCAATTGCTTTGAACTCGATTG
AGTTTGTTATGACCTTGGTCCAGATTGTCGTAGCCATACTAGTTGTGAAAGGAACAAAAGGTG
AATATCCAGAAGCAACATGGATCCTCGTCTATGCTTATGGTAGTATTGCCAATCTCCCTATTCTT
TGTTGGCGTTTCTGGCAGATCCGTCATCCTGTCTCACACTTAAGAATCAACCGAGTCATGTTTC
GTTTGAGGACGATATTTGAATGTTTCTTTGCGGGTTGGTTCGTGGTGTTCTTCTGGGTCTTTGT
TTGCAGCTCATCATCTCTAGACCATTCTAGTCAACTCTTCTGGTTATGTGTGGTTTTCCTTGTTTT
CGGTTGCATTCGATATGTGCTCCCTATGGTAATATGTGCAGCGACGTGTTGCTGTGTGTTTATG
ACGTTATGTTTTAACGCCGTTAACACTTGA
AT1G15630 CDS
Seq ID no 6 is:
ATGGAGAGCCCTTTGTTGACCAAGAGTGAGCACAACGTCGAAGACGTTACAGTCCATGGCGA
TCCTTCCTCAAATGAAGAGCACATAGTCGACATTACAACCAATGGAGATTCTTCACCAGCTGAT
GAGCACATCGTGGACATTACAATCAATGGCGATTCGTCATCAGCTGAAGAGCAAACTCCACAT
GAGGCTTCTCAATGGAGTTGTTCATTTATAAACGGAATTGTCTGGATTTTGGTCGAGTTAGCTG
TGACTTTGTCACAGATTGTTGCAGCCACATTTTTTCTGACTCTGACAAAAGATGAACAGCATCC
AGAACTAAATCATGTACCTTTGTTAATATGGATCATCTGCTATACTTGTGCCTCTATTGCCACTG
TACCTATTGTATGTTGCCGTTTATGGCAATATATCCGAACCGCTAGATCAGGGACATGTTACGA
GTTGATCGGTTTGGAAAATTTTCTTGAAACTTGCTTCGTCTGTTTGGTTGTGATGTTTTTGTTGG
GTTTTTTAACGGAGTTATCATCACGTGATCCTAGTACTAGTCAACACTTCTGGTTATGGCTGGC
TCTAATTGCTTTCAGTTGCATTCGATATCTGCTTCCTAATCTCACATGTGTCAAAGAGTGCTTCG
TGTGGCCTGTGCTTTTCCTTGTTAAACAACTTTGGGAAGGCATAATAGCAATGATTGATGCGTT
AACTGCACTCATTGGGCTCATCATTGCAATCATTGGGTACATCTTATTTTTGATCCTTTCAATCT
TTATGTACATCTGCGGATAA
AT1G15640 CDS
Seq ID no 7 is:
ATGGAGGGGCCTTTGTTGACTAAGAATGAGCACATCCTCGACGACGTTACAATAATCCATGGC
GATTCTTCCTCAAACGAAGAGCACATCGTCAACATTACAACCAATGTCGATTCTTCATCAGATG
ATGAGCAAACACAGGATGATTGTGGTGATCATTTGAGACGTATAATAGCTCTTTTTGTGGATG
AAGGGTGTGATGGATTTACATCTAGTCATGTTTGGGTTATGGTCGAGTTTGTTTTGACTTTGCT
CCAGGTTGTTGCAGCCATTGCTGTCCTGACTCTGACAAAAGATGAAACAGATCCACAGAAAGT
GTTTCGTACACTGATCATCTGTTATACCGGTGGCTGTATTGTCGTGCTCCTTATTCTAGGTTTGG
ATTTCTGGGATTATTGCGGAAGATTCTACGAGGTGATGGAGAATTTGAAAAAGATGCTTGACT
ATTTCTTCGTGGGTTGGGTTGTGGTGTTTTCATGGCATCTTATAAACAACTCATCATCTCCAGAT
AATGCTACACAACAGTACTGGTTATGTATGGCTTTCCTTGTTGTCAGTTGCATTCTACATGTGCT
TCCTAATCTCCCCTGCGCGGCGGCTTGTTTTCTATATCCTATGATACTTCGTCTTACCCAGTCTA
TAGACTTCTTTGATGACATTACTGAAAAGATAGAGGACATTAACTGGTTAATCTTTGTATACTT
TTTTGGAATCTTCTCCTGCATTATCTGTTGCATTTTTACTTGTTGCAGCAGATTATGTAGATAA
AT1G44010 CDS
Seq ID no 8 is:
ATGGAGGGGCCTTTGCTAACCAAGAACAAGCACATCAAAATCCATGGCGATTCTTCATCAAAT
GATGAGCATAATGTCAACATTACAATCACTAACGATTCTTCATCAACACCACACGGCGTTAATA
TTGTTGAACGGTGTATTCCATGCACAAGAGAAATGGAGTGGTTTTGTTTCATCGAGTTTGTTTT
GAATATTGTCCAAATTGTTGCAGCTTTTGTAGTTGTGACTCGGGCAAAAGATGAACATCCAGG
AACATCATTTCTTGTATGGATCATTGGTTACACTTGTGGATGTGTTGCCATTCTTCTTATTCAGT
TTATTAACCGTATTTCGTCAAGAAGCTACGAGGAGATCATGGACGTTTTGAAGAATATATGTG
AATATTTCTTCGTTGGTTGGGTTGTGTTGTTTCTTTGGATGTATCATAGTAGTTCTTCATCTCTA
TATGATAATACTCAATATTTCTGGTTATGTATGGCTTTTCTTGCTTTCACTTGCATTCGATATGTA
CCGGCTCGTTTAATATGTTCAGCAATATGCTTTATTTTCGTTGTGATAATATGGTTTTGCGCCGA
TGTGTTAGGCACAACCCCGAGTATGTTTGCTAATGCTGTATTCCTTATAGTTTTCATTGGAATTC
TTAAAGTCATTGAGGAGGTTATTGGATCCTATTGTTGTTGA
Seq ID no: 1 is the sequence of the SYAC1 gene.
AT1G15600
Seq ID no 9 is:
MEGPLLSECEHIIDITSDGSSSSIDGQQPHEGVELGDPYTNESICNSIEFVVTLVLIAAVIIVLIEPRDEE
HPQTMLSIWIIGYTCGCIATLPILCWRFWLYNRSVGSESTDEYLRETRINKDMDFFMTGFFVGWYV
VFLWILSSKAFDSSALDDTTTQYFWLCLALLTFSCIRYVLFNLTLAMVCYTTPLLLLTPVVICVLLAVEV
LKGIVACIVSCIC
AT1G15590
Seq ID no 10 is:
MDSESHSDNDDHIIDITNDEDSSSRSSLDERSYPSLSSSLSTDDESSDEGASSSTRDCGSLWNIMELV
VTLVQIVASLIVLTLAKDEHQQALLLTWVIGYTCGCITITLLILLSCVRKYNRIGVYSKTRTDRVMDAL
KMGNECFFVVWLVMGILWICYGHSSSSDTPKLYRFCKGRYRR
AT1G15610
Seq ID no 11 is:
MEGPLLTKSEHNVEDVTIHGDSSSNDEHIIDITINGGSSSADHEQTPHEAFQWSCPFTSGSFWILVE
LAVTLVQIVAAIFFLILTKDERDPELDVALFTWIIFYTCASIATLPFVCCRLWQYIQNASSETSNEVME
YRLENFLESFFVSLIVVSLWHILTESSSRQDTSRHFCCIRYVLPNFQFTVQLFMLPGVSLVTALVAAIV
TMINSLLSLFLVIIGLFALTFSWIIGMCG
AT1G15620
Seq ID no 12 is:
MEDPLLTQSEHIVDDVTIHGDSSSNEEHIVDVTTNGNPSSADEKRPHEGVQWSDIFTFTTVCILVEF
VVALVQIVAAIVVLTLAKDEQPPQKMFPTLILSYTGCCIATLPILGLRFWHSYRSVSTETRIYEVVDILK
KMLEYFFVGWVVVLLWHLINNSSSIDNTTQQFWLCMTFLAISCILHVLRNLPCAGVCFLYPMILYLS
QSIDFVGDITDEINLTTSIILLCFGIFACIICGCCSRCLCR
AT1G15625
Seq ID no 13 is:
MESPLLSNDDEHVDDMTADDYASLIYTLVSIPIAIALNSIEFVMTLVQIVVAILVVKGTKGEYPEAT
WILVYAYGSIANLPILCWRFWQIRHPVSHLRINRVMFRLRTIFECFFAGWFVVFFWVFVCSSSSLD
HSSQLFWLCVVFLVFGCIRYVLPMVICAATCCCVFMTLCFNAVNT
AT1G15630
Seq ID no 14 is:
MESPLLTKSEHNVEDVTVHGDPSSNEEHIVDITTNGDSSPADEHIVDITINGDSSSAEEQTPHEASQ
WSCSFINGVWLVELAVTLSQIVAATFFLTLTKDEQHPELNHVPLLIWIICYTCASIATVPIVCCRLW
QYIRTARSGTCYELIGLENFLETCFVCLVVMFLLGFLTELSSRDPSTSQHFWLWLALIAFSCIRYLLPN
LTCVKECFVWPVLFLVKQLWEGIIAMIDALTALIGLIIAIIGYILFLILSIFMYICG
AT1G15640
Seq ID no 15 is:
MEGPLLTKNEHILDDVTIIHGDSSSNEEHIVNITTNVDSSSDDEQTQDDCGDHLRRIIALFVDEGCD
GFTSSHVWVMVEFVLTLLQVVAAIAVLTLTKDETDPQKVFRTLIICYTGGCIVVLLILGLDFWDYCG
RFYEVMENLKKMLDYFFVGWVVVFSWHLINNSSSPDNATQQYWLCMAFLVVSCILHVLPNLPCA
AACFLYPMILRLTQSIDFFDDITEKIEDINWLIFVYFFGIFSCIICCIFTCCSRLCR
AT1G44010
Seq ID no 16 is:
MEGPLLTKNKHIKIHGDSSSNDEHNVNITITNDSSSTPHGVNIVERCIPCTREMEWFCFIEFVLNIVQ
IVAAFVVVTRAKDEHPGTSFLVWIIGYTCGCVAILLIQFINRISSRSYEEIMDVLKNICEYFFVGWVVL
FLWMYHSSSSSLYDNTQYFWLCMAFLAFTCIRYVPARLICSAICFIFVVIIWFCADVLGTTPSMFAN
AVFLIVFIGILKVIEEVIGSYCC

Seq ID no: 9 is the amino acid sequence of the SYAC1 protein.

Seq ID no: 9 is the amino acid sequence encoded by the nucleotide sequence of Seq ID no: 1.

Seq ID no: 10 is the amino acid sequence encoded by the nucleotide sequence of Seq ID no: 2.

Seq ID no: 11 is the amino acid sequence encoded by the nucleotide sequence of Seq ID no: 3.

Seq ID no: 12 is the amino acid sequence encoded by the nucleotide sequence of Seq ID no: 4.

Seq ID no: 13 is the amino acid sequence encoded by the nucleotide sequence of Seq ID no: 5.

Seq ID no: 14 is the amino acid sequence encoded by the nucleotide sequence of Seq ID no: 6.

Seq ID no: 15 is the amino acid sequence encoded by the nucleotide sequence of Seq ID no: 7.

Seq ID no: 16 is the amino acid sequence encoded by the nucleotide sequence of Seq ID no: 8.

Expression of the SYAC1 gene or a protein, or a gene or protein related thereto, may be under the control of the SYAC1 promoter. The whole or part of the promoter may be used.

The sequence of the SYAC1 promoter may have the sequence:

(SEQ ID No: 17)
TCCGGGAAGACCTAGCCGTAGTTCGGTGATTTCGAGTCCAAGATTAT
TACTCTCATTCGCCATCTTTGATGTTTTTGATTGAAAGTATTGGTGT
TAATGTGTTTGAACTTTATTCTGTTGCCACTTTATATGTTCTCATGG
GAACATAAACGATGGCGTTTGAGACAAGCTTGTTTAGCATTGATTTA
TCTTGTCGGCCCCATTCGAAGATCTTGTCGAAATTTTTATGTCATGT
TTTTTCATTCCTTTGCCGCTTCGTGTTTAAATGTCGATAATATGGAT
CCGTTGGTTCGACACACTGGACGGCCACGATTTTGTTACCGACAAAG
TAGGGTTTCTTTCTCAGCCGTTGGATCAGAAACACGTTACTTTCTAT
TAGACATCCCATGTTATGTGTGATTTGTTATGTCTGTATCCCATTCT
TTTAATCAACGACATAATAATAAAACAACATATTCATGGTCGAATAT
ATATTAGTGCACTTTAAAATCTACGTATATTTATGTTTCTCGACAAC
CGAACACGTGTTACTCAAGGCTAAGACAAGTTATTATATTATTTCAT
TTTTTTCTACACGTAACACACTACACAGTGCATACATAATTTCAAAG
AAAAAAATGCATCTACGTTTTCGTCATATATCTTGTTTCTGCTATTC
ACACATGTGCCCGATACTTTCATTACATCATACGTAATCTACACATG
TACACCATACTTTTGTCATTATTTGTTACGAAATAGAAACAGAACTG
ATCAAAATATCAATTACGGGATATATTTCCATTTTTCATAGATATTT
CCATATCTTTGTATCTCCAATAGTACATTTTTGGAATGTTCACATGT
TGATAGATAAGCTAGATTAGTCAAATTTCCTTGTACACAATTTCTAG
TTTTCGATTGATCATGTAGAATTGCGTATAATATATTGTCGGCTGGC
TTTAGCCAATGAATAACACAACAAGACTTTGTGATAATGGACAGCGA
ATCCCACAGTGATAATGATGATCACATAATCGACATTACAAACGATG
AAGATTCTTCATCAAGATCGTCTTTGGACGAGAGAAGCTATCCTTCA
TTAAGTTCGTCTCTGTCTACGGATGACGAATCGTCAGATGAAGGTGC
AAGTTCATCTACGAGAGACTGTGGTAGTCTTTGGAATATAATGGAAT
TAGTAGTCACTTTGGTTCAGATTGTTGCATCATTAATAGTTTTAACT
CTGGCAAAAGATGAACATCAACAAGCACTTTTGTTAACATGGGTCAT
CGGTTACACTTGTGGCTGTATTACCATTACGCTCCTTATTCTCCTCT
CGTGTGTCCGCAAATACAACCGAATTGGTGTTTACTCAAAAACGAGG
TATAATAAACAACACTTGTTTCACTTAACATTAAGAGTTTAATTAGC
TAAATCAAACCGAACCGTTGTTGGTGGCAGAACGGATAGGGTAATGG
ATGCTTTGAAGATGGGGAATGAATGTTTCTTTGTTGTGTGGTTAGTT
ATGGGGATTCTATGGATTTGTTATGGACACTCATCTTCTTCTGATAC
TCCTAAGCTCTACAGGTTTGACTCACAATCATGTCACAGTAGAGATT
TAGTTAAACCCTAACTAATCAGTACTAACTTTTGTCTTCTTGATTAT
GTTTTGGCAGGTTATGTGTAGTCTTCATTGCTTTCAGTTGCATTAGA
TTTGCATATGCATTACTATTATGCGCAGGTATTTTTTTCGCTTAATG
TTCACTAGAGTTTGGAACATTTTTTTCTCATATCTATGATGTGGATA
AGTTGAAATCAAATGATGTAGATTCTGTAAGGGAAGGTATAGGAGGT
AGATTTGTTTTCAAGAAACCGAGCCATGATGATGTGAGTAATAATTT
CATATCACATTAATTACTCTGTTTTAAATAGCATTTTTTTGTTGTTG
TTGTTGTTTAATTTTCATGGTTTGATGGTGTGTGCAGAGTTGTTGCA
TATGCTTGGGGAAATATGGAGAGGAAAAAGGAGTAGGACTAAGGAAA
CTTGAATGCTCACACGTATTTCATTCGCAATGTATAGACAAATGGCT
GAAAATCAAATCCACATGCCCTCTCTGTCAATCACAAGTTAGGTGGT
GATGAGATGAGGTTGATTTTTTTTTTATTTTTTTTTCGCTCAAGAGT
AATTGTACACATTCAATAAACAAAAGTGGTGGGAAACTGAAAGATAC
GATCCTGTCTTCTTGTGTTGTCTGTCTTAAAACAGAAAATAAATATC
ATACAGATCATTTGACCTATTAACAATTTAACTTGTCCAAAACATCT
GCAATATATATAAACATTGAAATCTCAGCTTTGATCTTCATTTTTCA
AAATAATGTTTTATTTGTAACACGACTAATACGTACTTAGTATTGAT
CTGGTCTAAAAAAATACTAATAATTTTATCAACAAAGAAGGAAAGGT
ATATGCTTTGTGCTCGTCAACGCAAATTTGAATGAACTTTTCATTTG
ATCGGCAAGAAATAGGAAAAACCAAAAGTGATCA

The annotated SYAC1 promoter sequence available from TAIR (The Arabidopsis Information Resource, https://www.arabidopsis.org) is 2525 bp upstream to the gene locus for SYAC1.

In a genetically modified plant or plant cell of the invention, SYAC1 gene expression can be modulated by the use of a naturally derived inducible promoter, such as the SYAC1 promoter, which is sensitive to specific hormones or compounds. In particular, the SYAC1 promoter is specifically sensitive to exogenously applied plant hormones: auxin and cytokinin. Moreover, elevated copper concentration in the plant medium (50 μM CuSO₄ in ½MS plus Agar substrate) is also able to trigger SYAC1 promoter-driven GUS (β-glucuronidase) expression in reporter Arabidopsis lines. Under elevated copper concentration, induction of gene expression by the SYAC1 promoter is possibly connected to impaired cell wall lignification which in turn affects the relative distribution of auxin and cytokinin in plant roots. Importantly, different promoter versions (i.e. in sequence length) showed different sensitivity and tissue localization in reporter gene expression in response to hormones or copper.

In an embodiment, the present invention uses a promoter of the sequence of SEQ ID NO: 17, or a sequence that has at least about 80%, 85%, 90%, 95%, 98%, 99% or more sequence identity with SEQ ID NO: 17, or a functional fragment thereof, to induce spatial and/or temporal expression of transgenes. The promoter may be used in brassicas or other plant species in response to hormones (such as auxin and/or a cytokinin) or elevated copper concentrations. The transgene may be SYAC1, alternatively the transgene may be any other transgene.

The plant may be any variety of plant species. The plant species may be a monocotyledonous plant. The monocotyledonous plant may be corn (Zea mays), sugar cane (Saccharum sp.), switchgrass (Panicum virgatum) and other grass species (Miscanthus), or any other monocotyledonous species used in bioethanol production. The present invention is also applicable in dicotyledonous plants, e.g. Arabidopsis. The plant may be a cereal, legume, fruit, root and tuber crop, oil crop, fibre crop or tree. The plant may be rice, wheat, barley, corn, maize, tomato, coffee plant, tobacco plant, tea plant, peanut plant, potato, carrot, fruit trees, oats, rye, soy bean, tricale, dry bean, mung bean, pea, lentil, banana, coconut, yarns, potato, sweet potato, cassava, sugar beet, cotton, jute, sesame, sunflower, rapeseed, or safflower.

Preferably a genetically modified plant or plant of the invention expresses the isolated nucleic acid so as to alter the phenotype of the plant compared to that of the non-genetically modified, wild type plant.

The expression of the isolated nucleic acid, such as the SYAC1 gene, or a related gene, in the genetically modified plant may result in one or more of the characteristics selected from the group consisting of a) an increased level of hemicellulose in the cell walls of at least some cells; b) less rigid cell walls in at least some cells; c) an increased tolerance to growth on contaminated soil; and d) an altered susceptibility to one or more microbes.

Reduced metal accumulation by a genetically modified plant on contaminated soil means that the plant can sustain productivity and quality more successfully on contaminated soil than a non-genetically modified, wild type plant.

An increase in the level of hemicellulose in plant cell walls may be defined as an at least about 20%, 30%, 40%, 50%, 60%, 70%, 80%, 90%, 100% or more increase in hemicellulose in the cell walls compared to that found in the cells walls of a wild type plant, or control plant. Preferably the wild type or control plant does not express the SYAC1 protein or a protein related to the SYAC1 protein, or does not express elevated levels of SYAC1 protein or a protein related to the SYAC1 protein.

A decrease in the rigidity of plant cell walls may be defined as an at least about 20%, 30%, 40%, 50% or more reduction in the median cell wall rigidity when compared to the cells walls of a wild type plant, or control plant. Preferably the wild type or control plant does not express SYAC1 protein or a protein related to the SYAC1 protein, or does not express elevated levels of SYAC1 protein or a protein related to the SYAC1 protein. Cell wall rigidity may be determined by using atomic force microscopy to calculate the apparent Young's modulus. By decreasing cell wall rigidity the efficiency of cell wall degrading enzymes is increased and thus so is the extraction of cell wall polysaccharides.

Genetically modified plants according to the invention may accumulate less heavy metals and minimize the negative effects on product quality caused by an excess of heavy metals in the soil.

An altered susceptibility to one or more microbes may result in a change in the colonisation efficiency of a particular microbe in a plant or plant cell of the invention. A plant or plant cell according to the invention with elevated levels of the SYAC1 protein or a protein related to the SYAC1 protein compared to a wild type plant or cell may be more susceptible to colonisation by beneficial microbes. A plant or plant cell according to the invention with reduced levels of the SYAC1 protein or a protein related to the SYAC1 protein compared to a wild type plant or cell may be more resistant to colonisation by harmful microbes, such as Plasmodiophora brassicae.

Genetically modified plants according to the invention may have a reduced level of, or no, expression of SYAC1 protein or a protein related to the SYAC1 protein and may display an altered sensitivity to metals compared to unmodified plants.

Preferably the level of SYAC1 protein expression or a protein related to the SYAC1 protein is not so high that it causes dwarfism in the plant.

In an embodiment, SYAC1 protein expression may be controlled only, or substantially only, in the root of a genetically modified plant.

Expression of SYAC1 in a cell type-specific and/or inducible way is possible by using a tissue specific promoter. Multiple inducible gene overexpression systems have been developed for plants and are described in Siligato, R., et al. (2016). MultiSite Gateway-Compatible Cell Type-Specific Gene-Inducible System for Plants1 [OPEN]. Plant Physiol. 170, 627-641.

According to a further aspect, the invention provides a promoter comprising or consisting of the sequence of SEQ ID NO: 17, or a sequence that has at least about 80%, 85%, 90%, 95%, 98%, 99% or more sequence identity with SEQ ID NO: 17, or a functional fragment thereof.

In a yet further aspect the invention provides a promoter comprising or consisting of the sequence of SEQ ID NO: 17, or a sequence that has at least about 80%, 85%, 90%, 95%, 98%, 99% or more sequence identity with SEQ ID NO: 17, or a functional fragment thereof for use in controlling expression of a transgene. Expression of the transgene by the promoter may be controlled by the presence of a hormone and/or a heavy metal. The hormone may be auxin and/or a cytokinin.

In a yet further aspect the invention provides a genetic construct comprising a promoter comprising or consisting of the sequence of SEQ ID NO: 17, or a sequence that has at least about 80%, 85%, 90%, 95%, 98%, 99% or more sequence identity with SEQ ID NO: 17, or a functional fragment thereof, operably linked to a transgene.

According to another aspect, the invention provides the use of a genetically modified plant or plant cell of the invention in the production of a bio fuel. The entire plant may be used or just a part thereof, for example the leaves or stem.

According to a further aspect, the invention provides the use of a genetically modified plant of the invention to produce crops on contaminated land more effectively or productively than the wild type plant.

According to a yet further aspect, the invention provides a seed produced by a genetically modified plant according to the invention.

According to a still further aspect the invention provides a method of producing a genetically modified plant, wherein the method comprises (a) transforming a plant cell with an isolated nucleic acid comprising or consisting of the sequence of Seq ID no: 1, Seq ID no: 2, Seq ID no: 3, Seq ID no: 4, Seq ID no: 5, Seq ID no: 6, Seq ID no: 7 or Seq ID no: 8, or a sequence that has at least about 80%, 85%, 90%, 95%, 98%, 99% or more sequence identity with one of Seq ID no: 1, Seq ID no: 2, Seq ID no: 3, Seq ID no: 4, Seq ID no: 5, Seq ID no: 6, Seq ID no: 7 or Seq ID no: 8, and (b) generating from the transformed cell a genetically modified plant that expresses the polypeptide encoded by the nucleic acid. Preferably the nucleic acid is operably linked to one or more regulatory sequences. The nucleic acid may be operably linked to: i) a promoter of Seq ID no: 17; ii) a functional fragment of the promoter of Seq ID no: 17; or a promoter having a 80%, 85%, 90%, 95%, 98%, 99% or more sequence identity with i) or ii). Preferably expression of the polypeptide results in a plant with one or more of a) an increased level of hemicellulose in the cell walls; b) less rigid cell walls; c) an increased tolerance to growth on contaminated soil; and d) an altered susceptibility to one or more microbes.

According to a further aspect the invention provides a method of producing a genetically modified plant comprising (a) transforming a plant cell with an isolated nucleic acid that encodes a polypeptide having at least about 80%, 85%, 90%, 95%, 98%, 99%, or 100% sequence identity with one of Seq ID no: 9, Seq ID no: 10, Seq ID no: 11, Seq ID no: 12, Seq ID no: 13, Seq ID no: 14, Seq ID no: 15 or Seq ID no: 16, and (b) generating from the transformed cell a genetically modified plant that expresses the polypeptide encoded by the nucleic acid. Preferably the nucleic acid is operably linked to one or more regulatory sequences. The nucleic acid may be operably linked to:

i) a promoter of Seq ID no: 17; ii) a functional fragment of the promoter of Seq ID no: 17; or a promoter having a 80%, 85%, 90%, 95%, 98%, 99% or more sequence identity with i) or ii). Preferably expression of the polypeptide results in a plant with one or more of a) an increased level of hemicellulose in the cell walls; b) less rigid cell walls; c) an increased tolerance to growth on contaminated soil; and d) an altered susceptibility to one or more microbes.

According to another aspect the invention provides a method of increasing the sugar content of plant cell walls, and/or altering the rigidity of plant cells and/or decreasing the uptake of heavy metals by a plant grown on soil contaminated with heavy metals, wherein the method comprises expressing within the plant an exogenous nucleic acid which encodes a polypeptide having at least about 80%, 85%, 90%, 95%, 98%, 99%, or 100% sequence identity with one of Seq ID no: 9, Seq ID no: 10, Seq ID no: 11, Seq ID no: 12, Seq ID no: 13, Seq ID no: 14, Seq ID no: 15 or Seq ID no: 16.

According to a further aspect, the invention also provides a genetically modified plant or plant cell which expresses an elevated level of SYAC1 protein or a protein related to the SYAC1 protein. The plant may be a brassica which naturally expresses SYAC1 protein or a protein related to the SYAC1 protein. The level of protein expression may be increased by 1 fold, 2 fold, 3 fold, 4 fold or more compared to a wild type, not modified, plant or plant cell. Alternatively, the level of protein expression may be increased by 20%, 30%, 40%, 50%, 60%, 70%, 80%, 90%, 100% or more compared to a wild type, not modified, plant or plant cell.

In another aspect the invention provides a genetically modified plant or plant cell which expresses a reduced level of, or no, SYAC1 protein or a related protein when compared to a wild type, not modified, plant or plant cell. Preferably the plant or plant cell expresses a reduced level, or no SYAC1, protein or a protein related to the SYAC1 protein, in the plant root or in a plant root cell. The level of SYAC1 protein or a related protein expression may be reduced by 10%, 20%, 30%, 40%, 50% or more compared to wild type, not modified, plant or plant cell. There may be no detectable SYAC1 protein or a related protein expression in the modified plant or plant cell. The modified plant or plant cell, expressing reduced or no SYAC1 protein or a related protein, preferably in the root, may display improved resistance to plant pathogens, in particular to Plasmodiophora brassicae which causes clubroot disease. Preferably the plant is from the Brassicaceae family, for example canola, rape, mustard, cabbage, broccoli or cauliflower.

Reference to the level of expression of the SYAC1 protein or a related protein may refer to the level of biologically active SYAC1 protein or a related protein in a cell or plant. Levels of SYAC1 protein or a related protein may be altered not only by altering the expression of the protein itself but by altering the level of expression of biological active SYAC1 protein or a related protein. For example, the introduction of mutations into the protein expressed may have the effect of altering the level of biologically active protein which has the effect of reducing the perceived level of expression of the SYAC1 protein or a related protein as referred to herein.

Reference herein to a genetically modified plant or cell includes a transgenic plant or a transgenic cell. Plants may be genetically modified by targeted mutagenesis, such as by using CRISPR-cas9 or by TILLING (Targeting Induced Local Lesions IN Genomes, or by random mutagenesis, such as can be achieved by UV or chemical mutagenesis.

In an embodiment, mutations may be induced in a plant or plant cell to produce a reduced level of expression of the SYAC1 protein or a related protein; this includes a reduced level of expression of biologically active SYAC1 protein or a related protein. This reduction in levels of expression of SYAC1 protein or a related protein may be used to alter the susceptibility of a plant or plant cell to colonisation by one or more microbes.

The skilled man will appreciate that any of the preferable features discussed above can be applied to any of the aspects of the invention or embodiments of the invention.

Preferred embodiments of the present invention will now be described, merely by way of example, with reference to the following figures and examples.

FIG. 1—is a simplified flow chart which shows the process of the production of bioethanol from plant material. Source: US DOE. June 2007. Biofuels: Bringing Biological Solutions to Energy Challenges, US Department of Energy Office of Science, reproduced with the permission of the US Department of Energy Genomic Science Program—https://genomicscience.energy.gov.

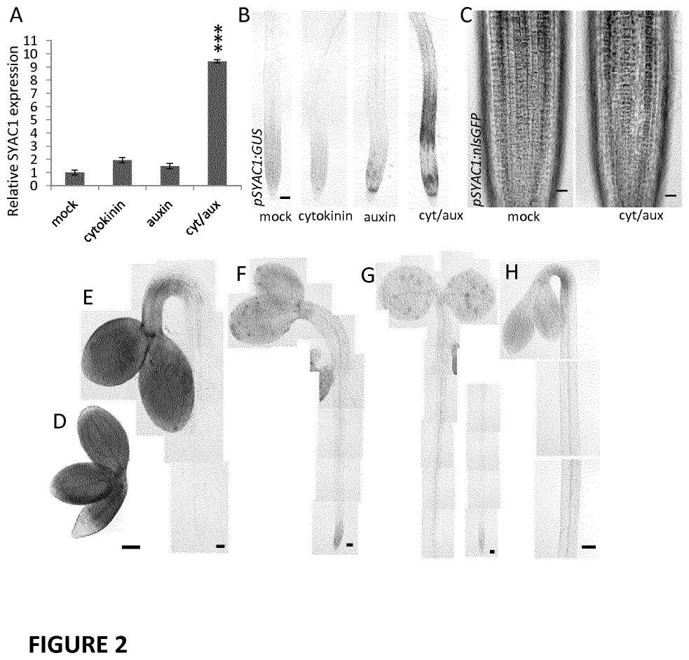

FIG. 2—shows the developmentally specific expression of SYAC1 and its regulation upon hormonal treatment. (A-C) Expression of SYAC1 in 5-day-old roots is synergistically upregulated after 6 hours treatment with 1 μM auxin and 10 μM cytokinin. SYAC1 expression in Arabidopsis roots monitored by RT-qPCR (A). Error bars represent standard error. Significant differences are indicated as ***P<0.001 (t test). Expression of pSYAC1:GUS (B) and pSYAC1:nlsGFP (C) upon hormonal treatment. (D-H) Expression pattern of SYAC1 from mature embryo till 4-day-old seedling. Mature embryo (D), 2, 3 and 4-day-old seedling (E-G); dark grown hypocotyl and apical hook of 3-day-old seedling (H). Scale bar 50 μm (B, E-G), 20 μm (C), 200 μm (D) and 100 μm (H). (A-C) Expression of SYAC1 in 5-day-old roots is synergistically upregulated after 6 hours treatment with 1 μM auxin and 10 μM cytokinin.

SYAC1 expression in Arabidopsis roots monitored by RT-qPCR (A). Error bars represent standard error. Significant differences are indicated as ***P<0.001 (t test). Expression of pSYAC1:GUS (B) and pSYAC1:nlsGFP (C) upon hormonal treatment. (D-H) Expression pattern of SYAC1 from mature embryo till 4-day-old seedling. Mature embryo (D), 2, 3 and 4-day-old seedling (E-G); dark grown hypocotyl and apical hook of 3-day-old seedling (H). Scale bar 50 μm (B, E-G), 20 μm (C), 200 μm (D) and 100 μm (H).

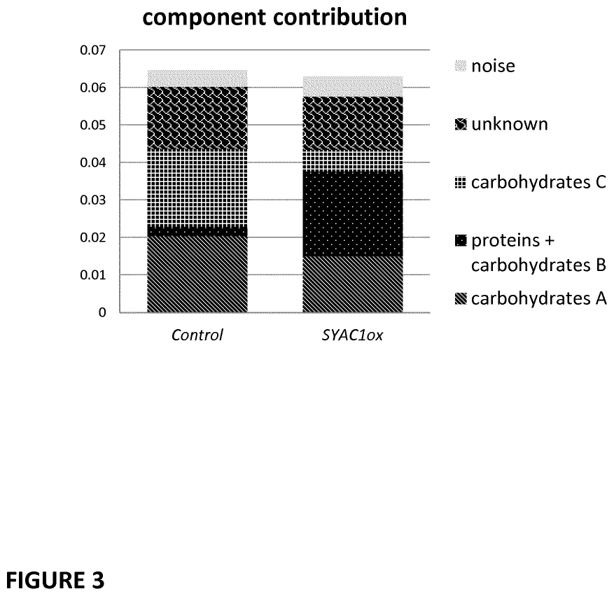

FIG. 3—shows the results of Fourier transform infrared spectroscopy (FT-IR) of 4 day old etiolated hypocotyls illustrating the differences in cell wall composition in control plant cells: Arabidopsis thaliana (L.) Heynh ecopyte Columbia and in SYAC1-HAox plant cells (plant cells transformed with the SYAC1 gene).

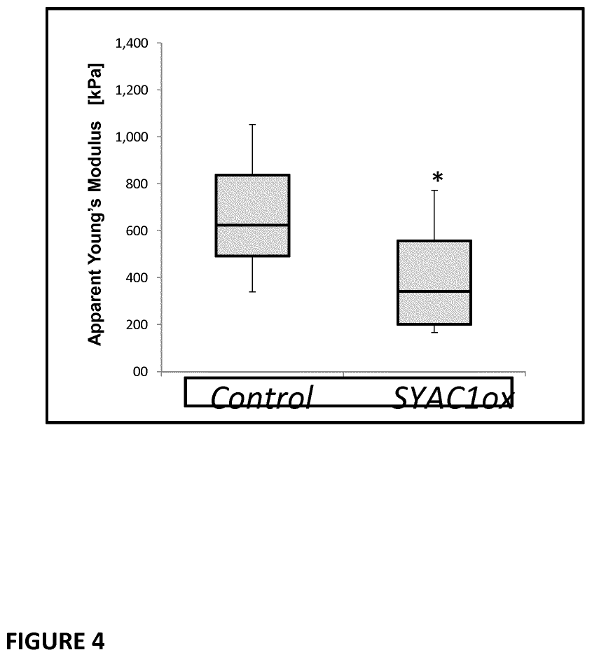

FIG. 4—shows the results of apparent Young modules measured by Atomic Force Microscopy in 4-day-old etiolated hypocotyls illustrating the differences in cell wall physical properties in control plant cells: Arabidopsis thaliana (L.) Heynh ecopyte Columbia and in SYAC1-GFPox plant cells (plant cells transformed with the SYAC1 gene). Significant differences are indicated as *P<0.05 (t-test).

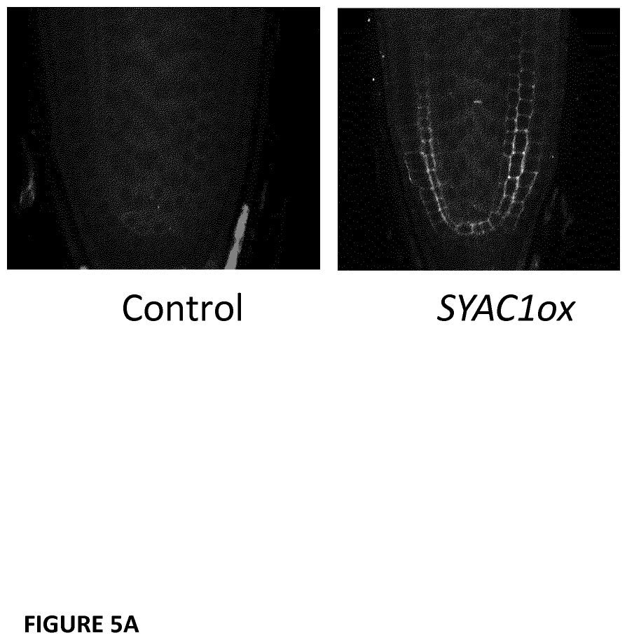

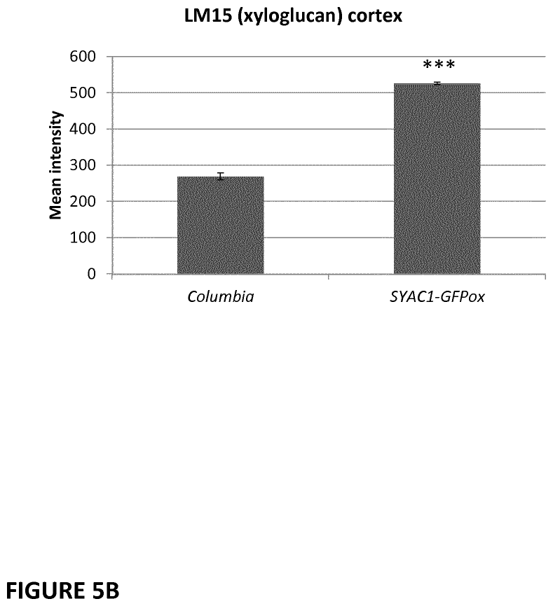

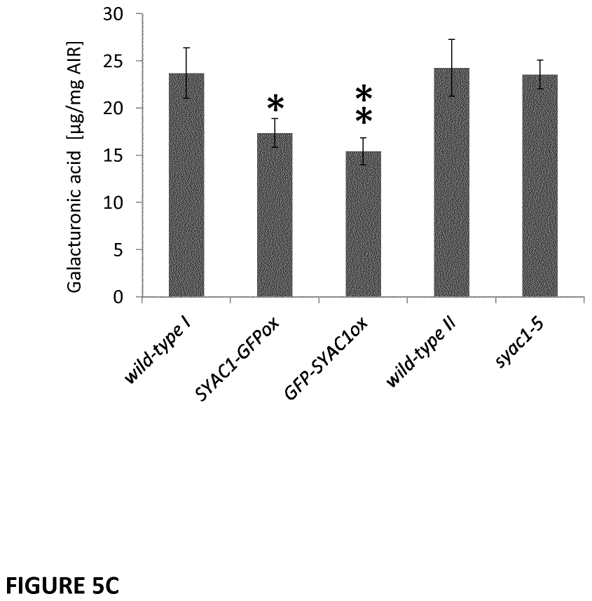

FIGS. 5A and 5B—shows the increase in hemicellulose concentration in the cell wall of plant cells expressing SYAC1 as determined by antibody staining. In particular, FIG. 5A shows the immunolocalisation of the hemicellulose xyloglucan using the LM 15 antibody and shows the change, and in particular, the increase, in xyloglucan localisation in the cell wall of cells of the SYAC1-GFPox line. FIG. 5B shows the quantification of xyloglucan immunodetected by LM 15 (xyloglucan antibody) in root meristem. Quantification was performed by measurement of membrane signal in cortex and epidermal cells, respectively. Signal in approximately 10 cells in a minimum of 10 roots was measured using ImageJ software. Statistical significance was evaluated by Student's t-test.

FIG. 5C shows that the amount of galacturonic acid (backbone of pectin structure) extracted from 4-day-old etiolated hypocotyls in SYAC1ox lines is significantly lower. (n=6, average±SE). Wild-type I represents corresponding control to SYAC-GFPox and GFP-SYAC1ox. Wild-type II was isolated from syac1-5 heterozygote population.

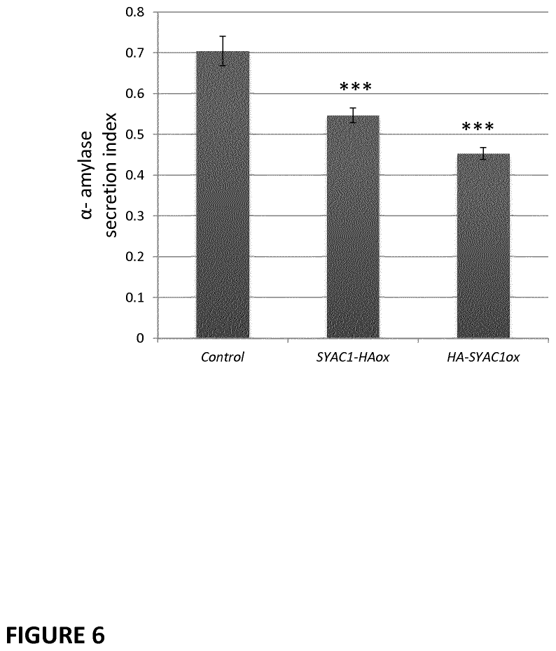

FIG. 6—shows the effect of SYAC1 expression on the α-amylase secretion index. The error bars indicate standard error calculated from 4 independent measurements. Significant differences are indicated as **P<0.01, and ***P<0.001 (t-test).

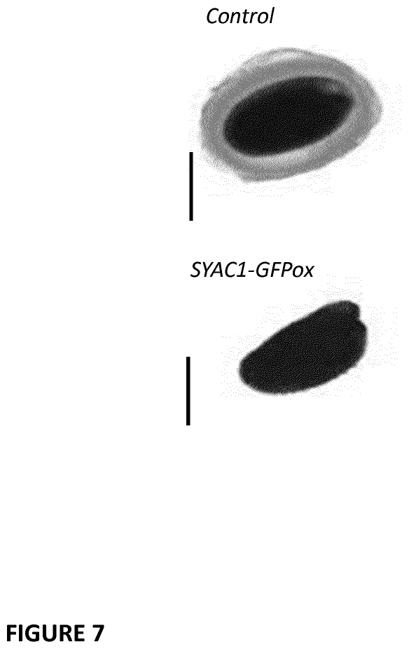

FIG. 7—shows the effect of SYAC1 expression on mucilage secretion. In particular, the figure uses ruthenium red to show that SYAC1 expression inhibits mucilage secretion. Scale bar 200 μm.

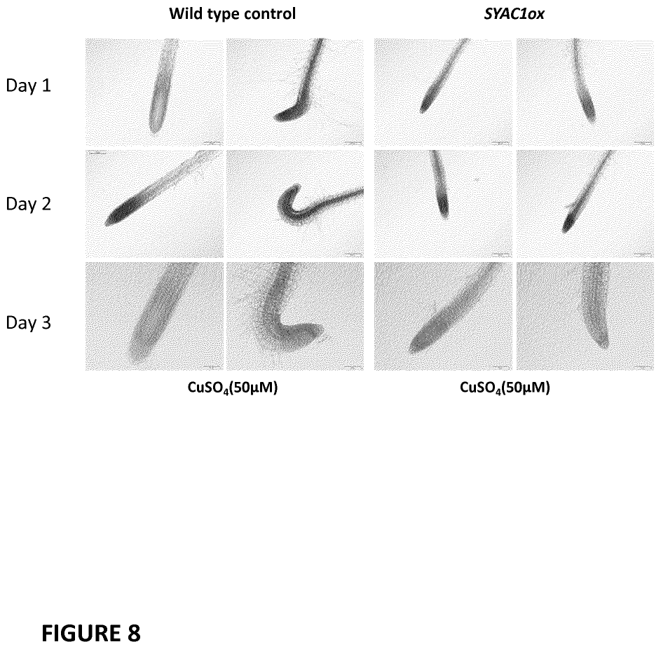

FIG. 8—shows that SYAC1 expression renders root plant more tolerant to elevated copper concentrations. Plants were grown for 5 days on standard MS media and then transferred to media containing 50 μM CuSO4. CuSO4 is shown to trigger swelling of cells at the root tip of wild type control plants, but not SYAC1-GFPox plants. These results demonstrate that SYAC1 expression renders plant cells more tolerant to elevated copper.

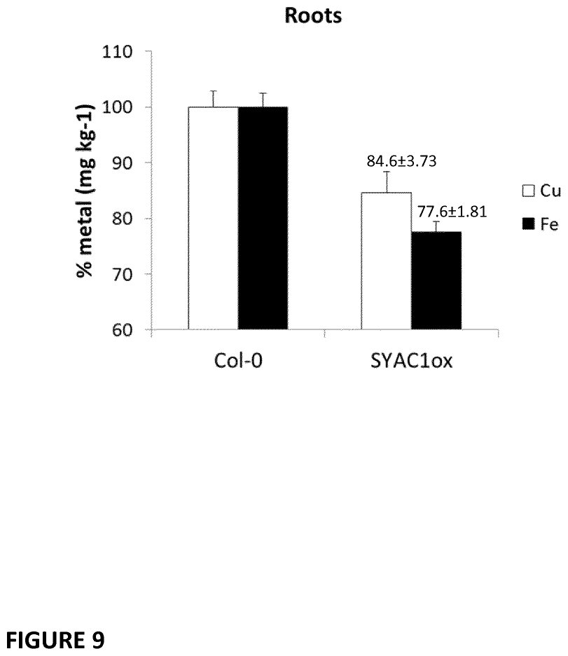

FIG. 9—shows that SYAC1 expression reduces the uptake of copper and iron. In particular, the results demonstrate that SYAC1 expression significantly reduces the amount of Cu or Fe taken up by plant roots. Wild type Col-0 and the SYAC1 overexpressor lines (HA-SYAC1ox) were grown for 1 week in standard MS media on agar plates. Fresh roots were collected and rinsed in deionized water during the sampling and stored in Falcon tubes. To remove the copper in the cell apoplast, fresh roots samples were immediately washed once in 40 ml 1 mM HCl solution (shaking end over end) for 3 mins. First washing solution was removed and samples were additionally washed in 0.01M HCl for 5 mins. Finally, root samples were rinsed in 40 ml deionized H₂O (Chaignon and Hinsinger, J. Environ. Qual., 2003). Samples were dried overnight in Falcon tubes at 65° C. in oven. For analysis of metal content, sample digestion has been performed at 150° C. for 4 h in a mixture of 65% HNO3 and 30% H2O2 in a ratio of 5:1 (v/v). Copper and iron were measured by detecting two isotopes, Cu-63 and Cu-65 or Fe-54 and Fe-57 respectively in a Perkin Elmer Elan DRCe 9000 (Rosenkranz et al., Waste Manag., 2018).

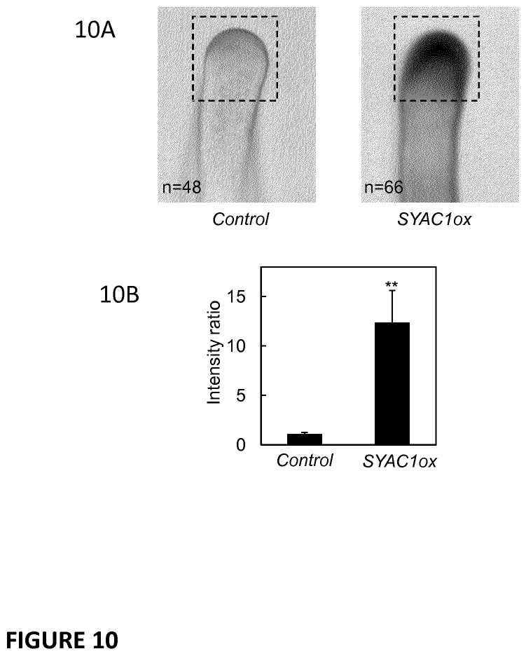

FIG. 10A—shows that SYAC1 expression is interfering with pectin secretion in tobacco pollen tubes (Nicotiana tabacum; ecotype Samsun N).

FIG. 10B shows the quantification of pectin detected by Ruthenium red staining in tobacco pollen tubes Nicotiana tabacum; ecotype Samsun N. Quantification was performed by measurement of ruthenium red intensity in the growing tips of tobacco pollen tubes. Signal in n=48 control pollen tubes and n=66 pollen tubes expressing SYAC1 was measured using ImageJ software. Statistical significance was evaluated by Student's t-test.

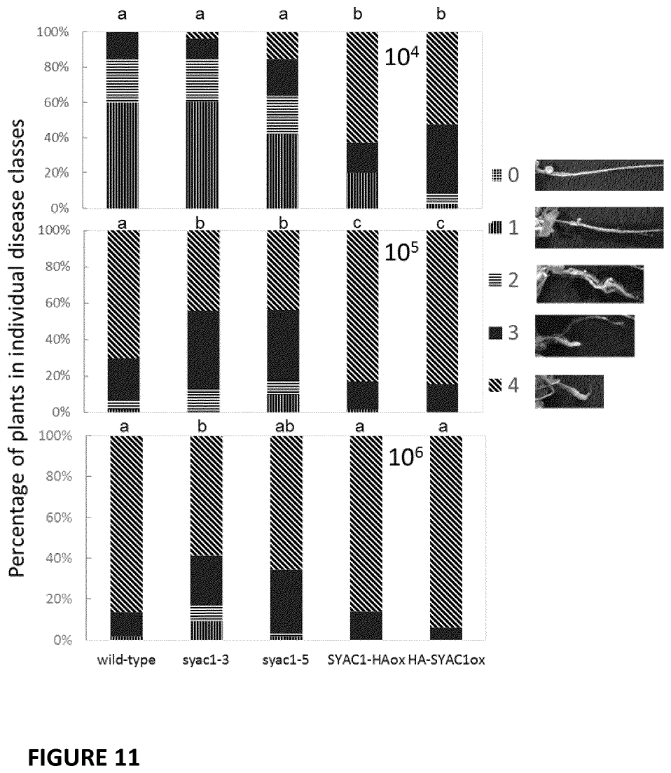

FIG. 11 shows the impact of SYAC1 on plant sensitivity to Plasmodiophora brassicae infection. Sensitivity of syac1-3 and syac1-5 mutant alleles, and SYAC1ox lines to pathogen infection. 5-scale classification was used to evaluate disease severity: 0 (no symptoms), 1 (very small galls mainly on lateral roots and that do not impair the main root), 2 (small galls covering the main root and few lateral roots), 3 (medium to large galls, also including the main root; plant growth might be impaired), and 4 (severe galls on lateral root, main root, or rosette; fine roots completely destroyed; plant growth is reduced). Inoculation was performed with 10⁴, 10⁵ and 10⁶ spores per mL. Significant differences between datasets are indicated by different letters.

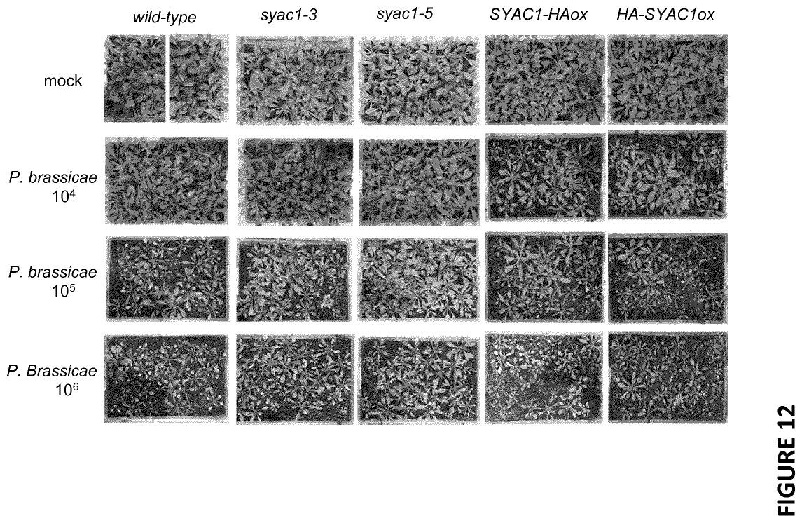

FIG. 12 also shows the impact of SYAC1 on plant sensitivity to Plasmodiophora brassicae infection. Shoots of wild-type, syac1-3, syac1-5 and SYAC1ox 28 days after P. brassicae inoculation are depicted. Inoculation was performed with 10⁴, 10⁵ and 10⁶ spore per mL.

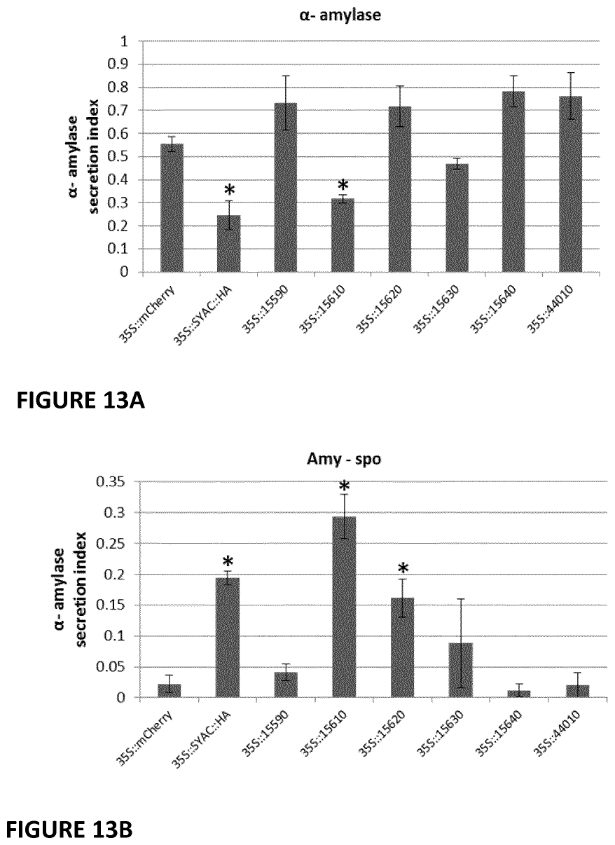

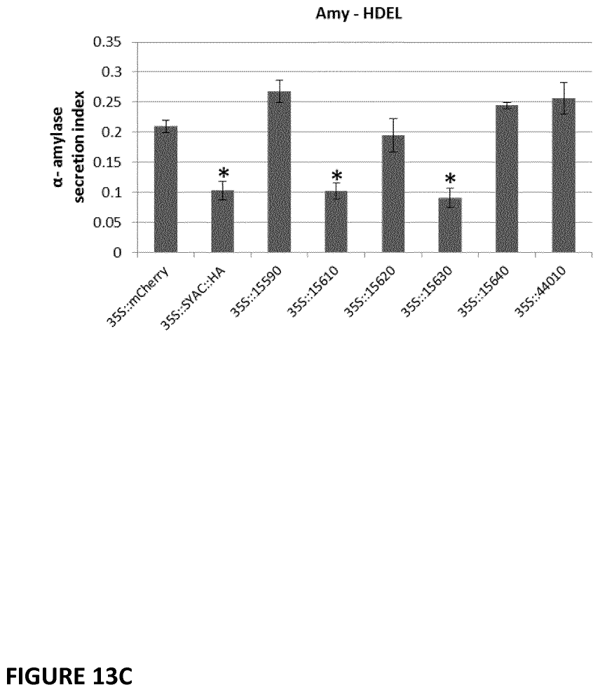

FIGS. 13A, 13B and 13C show the effect of the expression of SYAC1 paralogues on α-amylase secretion (the effect of SYAC1 is shown in FIG. 6). α-Amylase secretion assay was performed in Arabidopsis mesophyll protoplasts. SYAC1 and its paralogues were transiently co-expressed with α-Amylase (Amy) and α-Amylase derivatives fused to C-terminal vacuolar sorting (Amy-spo) and Endoplasmatic reticulum (ER) retention (Amy-HDEL) motif. FIG. 13A shows the effect of expression of SYAC1 and its paralogues on the α-amylase (Amy) secretion index. FIG. 13B shows the effect of expression of SYAC1 and its paralogues on the α-amylase fused to C-terminal vacuolar sorting motif (Amy-spo) secretion index. FIG. 13C shows the effect of expression of SYAC1 and its paralogues on the α-amylase fused to Endoplasmatic reticulum sorting motif (Amy-HDEL) secretion index. Secretion index was measured as a ratio of the α-Amylase activity in the medium and in the cells. The error bars indicate standard error calculated from 4 independent measurements. Significant differences are indicated as *P<0.05 (t-test).

### PLANT MATERIAL AND GROWTH CONDITIONS

Seeds of Arabidopsis were plated and grown on square plates with solid half strength Murashige and Skoog (MS) medium (Duchefa) supplemented with 0.5 g L⁻¹ MES, 10 g L⁻¹ Suc, 1% agar and pH adjusted to 5.9. The plates were incubated at 4° C. for 48 h to synchronize seed germination and then vertically grown under a 16:8 h day/night cycle photoperiod at 21° C.

The syac1-1 (salk_151420C, Col-0, KAN^{R}) and syac1-2 (salk_151662B, Col-0, KAN^{R}) T-DNA insertion lines were obtained from the Salk Institute. The syac1-3 (GABI-KAT 760F05, Col-0, SUL^{R}) and syac1-4 (GABI-KAT 961C03, Col-0, SUL^{R}) T-DNA insertion lines were obtained from the GABI KAT seed collection. The syac1-5 CRISPR line was prepared in collaboration with the VBCF Protein Technologies Facility (www.vbcf.ac.at) (see below). The genetically modified fluorescent-protein marker lines in Col-0 background have been described elsewhere: mCherry tagged wave line 6, 9, 13, 18, 25, 29, 34, 127, 129, 131, 138 (Geldner et al., 2009, Plant J. 59, 169-178), SYP61:SYP61-CFP (Drakakaki et al., 2012, Cell Res. 22, 413-424). The echidna mutant has been described in (Gendre et al., 2011, PNAS 108, 8048-8053) and yip4a-2 yip4b-1 in (Gendre et al., 2013, The Plant Cell 25, 2633-2646). Seeds of Arabidopsis were plated and grown on square plates with solid half strength Murashige and Skoog (MS) medium (Duchefa) supplemented with 0.5 g L⁻¹ MES, 10 g L⁻¹ Sucrose, 1% agar and pH adjusted to 5.9. The plates were incubated at 4° C. for 48 h to synchronize seed germination and then vertically grown under a 16:8 h day/night cycle photoperiod at 21° C. Cytokinin and auxin treatments were performed with the N6-benzyladenine cytokinin derivative (Sigma) and Naphthaleneacetic acid (Sigma), respectively. Short treatments (6 hours) for GUS/GFP expression were performed with 10 μM cytokinin and 1 μM auxin (unless indicated differently). For root growth transient assay 0.1 μM cytokinin and 0.05 μM auxin was used. Estradiol treatment was performed with β-Estradiol (Sigma).

Cloning and Generation of Genetically Modified Lines

All cloning procedure was conducted by using Gateway™ (Invitrogen) technology; with the sequences of all used vectors available online (https://gateway.psb.ugent.be/). To generate lines with constitutive overexpression of SYAC1 (SYAC1-GFPox, SYAC1-HAox, HA-SYAC1ox), SYAC1 ORF sequence was amplified and fused through a linker (4 Glycines and 1 Alanine) to GFP or HA tag. The fragments were first introduced into pDONR221 and then into pB2GW7,0 vector. All genetically modified plants were generated by the floral dip method (Clough and Bent, 1998, J. Cell Mol. Biol. 16, 735-743), and transformants were selected on plates with appropriate antibiotic.

For promoter analysis of SYAC1, an upstream sequence of 2522 bp was amplified by PCR and introduced into the pDONRP4-P1R entry vector. Then transcriptional lines (pSYAC1:GUS, pSYAC1:nlsGFP) were created: for pSYAC1:GUS, an LR reaction with SYAC1 promoter in pDONORP4-P1R, pEN-L1-S-L2,0 and pK7m24GW,0 vectors was performed. For pSYAC1:nlsGFP line, an LR reaction with SYAC1 promoter in pDONORP4-P1R, pEN-L1-NF-L2,0 and pB7m24GW,0 was performed. To generate overexpressor and inducible lines (SYAC1-GFPox, SYAC1-HAox, HA-SYAC1ox, pEST:SYAC1-GFP, pEST:SYAC1), SYAC1 ORF sequence with or without STOP codon was amplified and fused through a linker (4 Glycines and 1 Alanine) to GFP or HA tag. The fragments were first introduced into pDONR221 and then into pB2GW7,0 (overexpressor lines), p2GW7,0 (protoplast expression assays), pMDC7 (estradiol inducible line). For GFP-SYAC1ox genetically modified line SYAC1 ORF was amplified, introduced to pDONR221 and to the pB7FWG2.0 destination vector. To generate translational fusion line pSYAC1:gSYAC1-GFP, SYAC1 promoter was amplified together with the genomic fragment of the SYAC1 gene, cloned into pDONRP4-P1R and together with pEN-L1-F-L2,0 introduced into pB7m24GW,3. All genetically modified plants were generated by the floral dip method (Clough and Bent, 1998) in Columbia (Col-0) background and transformants were selected on plates with appropriate antibiotic.

Generation of CRISPR/Cas9 Line

Design of the gRNA for SYAC1 gene, molecular cloning and plant transformation was done in collaboration with VBCF Protein Technologies Facility (www.vbcf.ac.at). Design, specificity and activity of gRNA: GATGGTCAGCAACCACACGA (Seq ID no: 18) was performed using online available tools: http://cbi.hzau.edu.cn/cgi-bin/CRISPR and http://www.broadinstitute.org/rnai/public/analysis-tools/sgrna-design. gRNA was cloned into pGGZ003 CRISPR/Cas9 destination vector. Transformants resistant to an appropriate antibiotic were selected, genomic sequence of SYAC1 amplified and sequenced. Individual mutant lines with a single base pair insertion in the coding sequence (90 bps after the ATG—at the place of gRNA binding) were selected. Plants were propagated to obtain homozygote lines and CRISPR/Cas9 cassette was outcrossed.

Quantitative RT-PCR

RNA was extracted (RNeasy kit (Qiagen)) from roots of 6-day-old plants under all conditions (untreated, 1 μM auxin, 10 μM cytokinin and both together for 6 h). A DNase treatment with the RNase-free DNase Set (Qiagen) was carried out for 15 min at 25° C. Poly(dT) cDNA was prepared from 1 μg of total RNA with the iScript cDNA Synthesis Kit (Biorad) and analyzed on a LightCycler 480 (Roche Diagnostics) with the SYBR Green I Master kit (Roche Diagnostics) according to the manufacturer's instructions. SYAC1 expression was quantified with specific primer pair Fw: ACTTCTGGTTATGTTTGGCTCTCC (Seq ID no: 19) and Rv: ACACATATGACCACAGGCGTAAG (Seq ID no: 20). All PCRs were performed in 3 technical replicates and the experiments were repeated three times with similar results. Expression levels were first normalized to CDKA1 (Fw: ATTGCGTATTGCCACTCTCATAGG (Seq ID no: 21) and Rv: TCCTGACAGGGATACCGAATGC (Seq ID no: 22)) expression levels and then to the respective expression levels in untreated or in wild-type plants.

Phenotypic Analysis

For root and hypocotyl length analysis, seedlings were photographed and lengths were measured with ImageJ software (https://imagej.nih.gov/ij/). About 20-30 seedlings were processed and 3 independent experiments were performed.

Histochemical and Histological Analysis

To detect β-Glucuronidase (GUS) activity, mature embryos and 2 to 4-day-old seedlings were incubated in reaction buffer containing 0.1M sodium phosphate buffer (pH 7), 1 mM ferricyanide, 1 mM ferrocyanide, 0.1% Triton X-100 and 1 mg m1⁻¹ X-Gluc for 12 h in dark at 37° C. Afterwards, chlorophyll was removed by destaining in 70% ethanol and seedlings were cleared as described (Malamy and Benfey, 1997, Development 124, 33-44). In brief, seedlings were incubated in a solution containing 4% HCl and 20% methanol for 10 min at 65° C., followed by 10 min incubation in 7% NaOH/60% ethanol at room temperature. Next, seedlings were rehydrated by successive incubations in 60, 40, 20 and 10% ethanol for 15 min, followed by incubation (15 min up to 2 h) in a solution containing 25% glycerol and 5% ethanol. Finally, seedlings were mounted in 50% glycerol. GUS expression was monitored by differential interference contrast microscopy (Olympus BX53).

Immuno labeling in roots (4 to 5-day-old seedlings) was performed using an automated system (Intavis in situ pro) according to published protocol (Sauer et al., 2006, Nature Protocols. Nat. Protocols 1, 98-103). Roots were fixed in 4% paraformaldehyde for 1 h in vacuum at room temperature. Afterwards, seedlings were incubated for 30-45 min in PBS (2.7 mM KCl, 137 mM NaCl, 4.3 mM Na₂HPO₄2H₂O and 1.47 mM KH₂PO₄, pH 7.4) containing 2% driselase (Sigma), and then in PBS supplemented with 3% NP40 and 20% DMSO. After blocking with 3% BSA in PBS, samples were incubated with primary antibody for 2 hours. Antibody dilutions were rabbit anti-BIP2 (1:200) (Agrisera AS09481), rabbit anti-SEC21 (1:800) (Agrisera AS08327), rabbit anti-ARF1 (1:600) (Agrisera AS08325), rabbit anti-SYP61 (1:200) (Sanderfoot et al., 2001), rabbit anti-ECH (1:600) (kindly provided by R. P. Bhalerao, Umea Plant Science Centre), rabbit anti-ARA7+RHA1 1:1 (1:100) (Haas et al., 2007), rabbit anti-PIN1 (1:1000) (Paciorek et al., 2005), rabbit anti-PIN2 (1:1000) (provided by C. Luschnig, University of Natural Resources and Life Sciences, Vienna) and mouse anti-GFP (1:600) (Sigma G6539). Secondary antibody incubation was carried on for 2 h. Anti-mouse-Alexa 488 (Life Technologies, 1252783) and Cy3-conjugated anti-rabbit antibody (Sigma, C2306) were diluted 1:600 in blocking solution. Samples were mounted in solution containing 25 mg mL⁻¹ DABCO (Sigma) in 90% glycerol, 10% PBS, pH 8.5. Signal was monitored using a confocal laser scanning microscope (LSM 700, Zeiss). Images were analyzed by using ImageJ software.

Co-Localization Analysis

Pearson's correlation coefficient (R) was used for co-localization analyses: the analysis is based on the pixel intensity correlation over space and was performed using Image J software. After splitting the two channels, region of interest (ROI) was identified. For this analysis, 1 cell was considered as 1 ROI; in every root approximately 5 cells (5 ROIs) were measured and a minimum of 10 roots were used. Co-localization plug-in using an automatic threshold was used to obtain Rcoloc value, which represent Pearson's correlation coefficient.

Confocal Imaging and Image Analysis

Zeiss LSM 700 confocal scanning microscope using either 20× or 40× (water immersion) objectives were employed to monitor expression of fluorescent reporters. GFP (YFP) and Cy3 signals were detected either at 488 nm excitation/507 nm emission or 550 nm excitation/570 nm emission wavelength, respectively. Quantification of immunodetected PIN1 and PIN2 expression in root meristems was performed by measurement of membrane signal in cortex and epidermal cells, respectively. Signal in approximately 10 cells in a minimum of 10 roots was measured using ImageJ software. Statistical significance was evaluated by Student's t-test.

Transient Expression in Root Suspension Culture Protoplasts

The transient expression assays were performed on 4-days-old Arabidopsis root suspension culture by PEG mediated transformation. Protoplasts were isolated in enzyme solution (1% cellulose (Serva), 0.2% Macerozyme (Yakult), in B5-0.34M glucose-mannitol solution (2.2 g MS with vitamins, 15.25 g glucose, 15.25 g mannitol, pH to 5.5 with KOH) with slight shaking for 3-4 h, and afterwards centrifuged at 800 g for 5 min. The pellet was washed and resuspended in B5 glucose-mannitol solution to a final concentration of 4×10⁶ protoplasts/mL. DNAs were gently mixed together with 50 μL of protoplast suspension and 60 μL of PEG solution (0.1M Ca(NO₃)₂, 0.45M Mannitol, 25% PEG 6000) and incubated in the dark for 30 min. Then 140 μL of 0.275M Ca(NO₃)₂ solution was added to wash off PEG, wait for sedimentation of protoplasts and remove 240 μLof supernatant. The protoplast pellet was resuspended in 200 μL of B5 glucose-mannitol solution and incubated for 16 h in the dark at room temperature. Transfected protoplasts were mounted on the slides and viewed with Zeiss LSM 700 confocal scanning microscope.

Transient Expression in Arabidopsis Mesophyll Protoplasts

Mesophyll protoplasts were isolated from rosette leaves of 4-week-old Arabidopsis plants grown in soil under controlled environmental conditions in a 16:8 h light/dark cycle at 21 C. Protoplasts were isolated and transient expression assays were carried out as described (Wu et al., 2009, Plant Methods 5, 16).

Transient Expression in Tobacco Pollen Tubes

SYAC1 was transiently expressed in tobacco (Nicotiana tabacum) pollen tubes under a pollen-specific Lat52 promoter, exactly as previously described (Ischebeck et al., 2008, Plant Cell 20, 3312-3330).

Co-Immunoprecipitation (Co-IP) Assays.

For the Co-IP assays, proteins were expressed in root suspension culture protoplasts (see above) and extracted from the cell pellet as described previously (Cruz-Ramirez et al., 2012, Cell 150, 1002-1015); vectors containing ECH-HA and YIP4a-Myc were kindly provided by R. P. Bhalerao, Umea Plant Science Centre. 100 μg total protein extract was incubated in a total volume of 100 μL extraction buffer containing 150 mM NaCl and 1 μg anti-GFP (JL-8, Clontech) or 1.5 μg anti-cMyc (clone 9E10, Covance). After 2 h, 15 μL ProteinG-Magnetic Beads (BIO-RAD), which were previously equilibrated in TBS buffer we added and this mixture was further incubated for another 2 h on a rotating wheel at 4° C. The beads were then washed in 3×500 μL washing buffer (1×TBS, 5% glycerol, 0.1% Igepal CA-630) and eluted by boiling in 25 μL 1.5× Laemmli sample buffer. Proteins were then resolved with SDS-PAGE and blotted to PVDF transfer membrane (Millipore). The presence of the proteins of interest was tested by immunodetection using rat anti-HA-peroxidase (3F10, Roche).

Bimolecular Fluorescence Complementation (BiFC) Assay

To generate constructs for BiFC assay, the ORFs for SYAC1, YIP4a, YIP4b, YIP5b, ECH, KCR1 and PHB4 proteins were cloned into the pDONRZeo vector. Next, the ORFs were transferred from their respective entry clones to the gateway vector pSAT4-DEST-n(174)EYFP-C1 (ABRC stock number CD3-1089) or pSAT5-DEST-c(175-end)EYFP-C1 (ABRC stock number CD3-1097), which contained the N-terminal 174 amino acids of enhanced yellow fluorescent protein (EYFP^{N}) or the C-terminal 64 amino acids of EYFP (EYFP^{C}), respectively. The fusion constructs encoding cEYFP-SYAC1 and nEYFP-YIP4a, nEYFP-YIP4b, nEYFP-YIP5b, nEYFP-ECH, nEYFP-KCR1 or nEYFP-PHB4 proteins were mixed at a 1:1 ratio and transfection of root suspension culture protoplasts (see above) was performed. SYAC1 in P2YGW7 was used as a positive control.

Yeast Two-Hybrid Assays.

Yeast two-hybrid assay was performed using the GAL4-based two-hybrid system (Clontech). Full-length SYAC1 and YIP4a, YIP4b, YIP5b, ECH, KCR1, DSK2, PHB4 ORFs were cloned into pDEST-GADT7 and pDEST-GBKT7 (Clontech) to generate the constructs AD-SYAC1 and BD-YIP4a (YIP4b, YIP5b, ECH, KCR1, DSK2, PHB4). The constructs were transformed into the yeast strain PJ69-4A with the lithium acetate method. The yeast cells were grown on minimal medium (-Leu/-Trp), and transformants were plated (minimal medium, -Leu/-Trp/-His without or with increasing concentration of 3-Amino-1,2,4-trizol) to test the protein interactions.

α-Amylase Enzymatic Assay.

α-Amylase assays and calculations of the secretion index were performed as described (Früholz and Pimpl, 2017, In Plant Protein Secretion, (Humana Press, New York, N.Y.), pp. 171-182); α-Amylase expression constructs were kindly provided by P. Pimpl and transfections were performed in Arabidopsis mesophyll protoplasts (see above). α-Amylase activity was measured with a kit Ceralpha (Megazyme). The reaction was performed in a microtiter plate at 37° C. with 30 μL of extract and 30 μL of substrate. The reaction was stopped by the addition of 150 mL of stop buffer. The absorbance was measured at a wavelength of 405 nm. Experiment was performed three times with four replicates.

Real-Time Analysis and Statistics of the Apical Hook Development

Development of seedlings was recorded at 1-h intervals for 5 days at 21° C. with an infrared light source (880 nm LED; Velleman, Belgium) by a spectrum-enhanced camera (EOS035 Canon Rebel Xti; 400DH) with built-in clear wideband-multicoated filter and standard accessories (Canon) and operated by EOS utility software. Angles between hypocotyl axis and cotyledons were measured by ImageJ software. At least 10 seedlings with synchronized germination were processed and the experiment was repeated three times with similar results. For more details see (Zhu et al., 2017, In Plant Hormones, J. Kleine-Vehn, and M. Sauer, eds. (New York, N.Y.: Springer New York), pp. 1-8).

AFM Measurements and Apparent Young's Modulus Calculations

The AFM data were collected and analyzed as described elsewhere with minor changes (Peaucelle et al., 2015 Curr. Biol. CB 25, 1746-1752). To examine extracellular matrix properties the turgor pressure was suppressed by immersing seedling in a hypertonic solution (0.55 M mannitol). 4 days-old seedlings grown in darkness were placed on petri dishes filled with 1% Agarose and 10% Mannitol and immobilized by low melting agarose (0.7% with 10% Mannitol). The focus on the atomic force microscope was set on the anticlinal (perpendicular to the organ surface) cell walls and its extracellular matrix. To ensure proper indentations (especially in the bottom of the dome shape between two adjacent cells regions), cantilevers with long pyramidal tip (14-16 μm of pyramidal height, AppNano ACST-10), with a spring constant of 7.8 N/m were used. The instrument used was a JPK Nano-Wizard 3.0 and indentations were kept to <10% of cell height. Three scan-maps per sample were taken over an intermediate region of the hypocotyls, using a square area of 25×25 μm, with 16×16 measurements, resulting in 1792 force-indentation experiments per sample. The lateral deflection of the cantilever was monitored and in case of any abnormal increase the entire data set was discarded. The apparent Young's modulus (EA) for each force-indentation experiment was calculated using the approach curve (to avoid any adhesion interference) with the JPK Data Processing software (JPK Instruments AG, Germany). To calculate the average EA for each anticlinal wall, the total length of the extracellular region was measured using masks with Gwyddion 2.45 software (at least 20 points were taken in account). The pixels corresponding to the extracellular matrix were chosen based on the topography map. For topographical reconstructions, the height of each point was determined by the point-of-contact from the force-indentation curve. A total of 12-14 samples were analyzed. A standard t-test was applied to test for differences between genotypes.

Ruthenium Red Staining

Mature seeds were incubated in 0.01% (w/v) aqueous solution of Ruthenium red. Seeds were mounted in water and viewed using a DIC Olympus BX53 microscope. Nicotiana tabacum pollen tubes were stained as previously described (Ischebeck et al., 2008, Plant Cell 20, 3312-3330)

Tandem Affinity Purification (TAP)

Tandem affinity purification assay was performed in Arabidopsis cell suspension culture as described (Van Leene et al., 2015, Nature Protocols 10, 169-187) with minor modifications. Briefly, SYAC1 was produced as N-terminally tagged GS^{TEV} fusion in PSB-D cell culture. Extract and binding were performed with 1% digitonin added to the standard buffer. Protein interactors were identified by mass spectrometry using an LTQ Orbitrap Velos mass spectrometer. Proteins with at least two matched high confident peptides were retained. Background proteins were filtered out based on frequency of occurrence of the co-purified proteins in a large dataset containing 543 TAP experiments using 115 different baits.

Cell Wall Analyses

Analyses were performed on 4 days old dark grown hypocotyls using an alcohol-insoluble residue (AIR) prepared as follows. Seeds of Arabidopsis were plated and grown on square plates with Milieu Arabidopsis medium (Duchefa) supplemented with 0.32 g L⁻¹ CaNO₃, 10 g L⁻¹ Suc, 0.8% agar and pH adjusted to 5.75. The plates were incubated at 4° C. for 48 h to synchronize seed germination and then vertically grown in darkness at 18° C. Freshly collected samples were submerged into 96% ethanol, grinded and incubated for 30 min at 70° C. The pellet was then washed twice with 96% ethanol and once with acetone. The remaining pellet of alcohol insoluble residues (AIR) was dried in a fume hood overnight at room temperature. Dry weight of each sample was measured. After saponification of the AIR (1-4 mg) with 0.05 M NaOH, supernatant containing methyl ester released from the cell wall was separated from the pellet with polysaccharides. Pectins were extracted from the pellet with 1% ammonium oxalate at 80° C. for two hours as described (Krupková et al., 2007, Plant J. 50, 735-750; Mouille et al., 2007, Plant J. 50, 605-614; Neumetzler et al., 2012, PLoS ONE 7, e42914). Galacturonid acid was then quantified by colorimetry using meta-hydroxydiphenyl-sulfuric acid method as described (Blumenkrantz and Asboe-Hansen, 1973, Analytical Biochemistry 54, 484-489). Methyl ester was quantified from NaOH supernatant with a colorimetric assay using enzymatic oxidation of methanol (Klavons and Bennett, 1986, J. Agric. Food Chem. 34, 597-599). The monosaccharide composition of the non-cellulosic fraction was determined by hydrolysis of 100 μg AIR with 2 M TFA for 1 h at 120° C. After cooling and centrifugation, the supernatant was dried under a vacuum, resuspended in 200 μL of water and retained for analysis. All samples were filtered using a 20-μm filter caps, and quantified by HPAEC-PAD on a Dionex ICS-5000 instrument (ThermoFisher Scientific) as described (Fang et al., 2016, The Plant Cell 28, 2991-3004).

Fourier Transform Infrared Spectroscopy (FT-IR)

Spectra were recorded from the 4 days old dark grown hypocotyls sections in transmission mode on a Bruker Tensor 27 spectrometer equipped with a Hyperion 3000 microscopy accessory and a liquid N₂ cooled 64×64 mercury cadmium telluride (MCT) focal plane array (FPA) detector. The entire setup was placed on a vibration-proof table. Spectra were recorded in the region 900-3900 cm⁻¹, with 4 cm⁻¹ spectral resolution and 32 scans co-added in double sided, forward-backward mode. FPA frame rate was 3773 Hz and integration time 0.104 ms, with offset and gain optimized for each sample between 180-230 and 0-1, respectively. A low pass filter and an aperture of 6 mm were used. 4 hypocotyls for each line were used and 5 spectra from each of 3 different regions were used in the analyses. Background was recorded on a clean, empty spot on the CaF₂ carrier (Crystran Ltd, UK) and automatically subtracted. Fourier transformation was carried out using a zero filling factor of two, and Blackman-Harris 3-term apodization function. Phase correction was set to the built-in Power mode with no peak search and a phase resolution of 32. White light images were recorded with a Sony ExwaveHAD color digital video camera mounted on the top of the microscope and exported as jpg files. Spectra were recorded using OPUS (version 6.5 and 7, Bruker Optics GmbH, Ettlingen, Germany), cut to the fingerprint region of 900-1800 cm⁻¹ and exported as .mat files for subsequent processing and analysis. The exported spectra were pre-processed by an open-source software developed at the Vibrational Spectroscopy Core Facility in Umeå (https://www.umu.se/en/research/infrastructure/visp/downloads/), written in MATLAB (version 2014a-2018b, Mathworks, USA), using asymmetric least squares baseline correction (Eilers, 2004); (lambda: 100,000 and p=0.001), Savitzky-Golay smoothing (Savitzky and Golay, 1964); using a 1st order polynomial, with a frame number of 5; and total area normalization. Multivariate Curve Resolution—Alternating Least Squares (MCR-ALS) analysis was performed on the spectra using 5 components (based on singular value decomposition of the initial dataset). A maximum of 50 iterations and a convergence limit of 0.1 were used, with initial estimates in the spectrum direction (determined automatically by the built-in SIMPLISMA based algorithm) and a noise level of 10% given in the script. Only non-negativity constraints were used, both in the spectrum and concentration dimensions. The resulting profiles explained 99.84% of the variation. For classification, k-means clustering was performed within this open-source software, using the resolved spectral profiles and MATLAB's built-in algorithm.

Clubroot Infection Rating

All experiments were performed with the Plasmodiophora brassicae single-spore isolate e3 (Fähling et al., 2003, Journal of Phytopathology 151, 425-430) and Arabidopsis thaliana ecotype Columbia was used as the wild-type. Resting spores were extracted by the homogenization of mature clubroot galls (stored at −20° C.) of Chinese cabbage, followed by filtration through gauze (25-mm pore width) and two centrifugation steps (2,500×g, 10 min). Fourteen-day-old Arabidopsis seedlings, which were cultivated in a controlled environment (23° C., 16-h light, 100 mmol photons/s/m2) using a compost:sand (9:1 v/v) mixture (pH 5.8), were inoculated by injecting the soil around each plant with 1 ml of a resting spore suspension in Na₂HPO₄ buffer (pH 5.8), with the spore concentration 10⁶, 10⁵ and 10⁴ spores per ml. Controls were the same age and were treated with Na₂HPO₄ buffer (pH 5.8) (mock) instead of spore suspension. Disease symptoms were assessed at 28 days after inoculation (dai). At least 30 Arabidopsis plants were analyzed for each line and treatment. The disease severity was assessed qualitatively on the basis of the infection rate and a disease index (DI) as described by Siemens et al., (2002) using the following 5-scale classification: 0 (no symptoms), 1 (very small galls mainly on lateral roots and that do not impair the main root), 2 (small galls covering the main root and few lateral roots), 3 (medium to large galls, also including the main root; plant growth might be impaired), and 4 (severe galls on lateral root, main root, or rosette; fine roots completely destroyed; plant growth is reduced). Data are displayed as percentage of plants in the individual disease classes since this gives a more detailed view on the differences. Data presented are means of two independent experiments for the 10⁶ and 10⁵ spore concentrations and one for the 10⁴ spore concentration. The qualitative disease assessment data were analyzed using the Kruskal-Wallis-test and by subsequently comparing the mean rank differences as described in Siemens et al., (2002, Journal of Phytopathology 150, 592-605).

Accession Numbers

Sequence data relating to this invention can be found in GenBank/EMBL data libraries under the following accession numbers: SYAC1, At1 g15600; YIP5b, At3 g05280; YIP4a, At2 g18840; YIP4b At4 g30260; ECH, At1 g09330; KCR1, At1 g67730; DSK2, At2 g17200; PHB4, At3 g27280.

Results

Auxin and Cytokinin Synergistically Control Expression of SYAC1 in Root.

To search for novel molecular components and mechanisms of auxin-cytokinin cross-talk, genome wide transcriptome profiling of roots exposed to auxin, cytokinin and both hormones simultaneously, was performed. SYNERGISTIC AUXIN CYTOKININ 1 (SYAC1, AT1G15600), which encodes a protein of unknown function, was identified as a gene whose expression was synergistically up-regulated by simultaneous hormonal treatment when compared to the expected additive effect of both hormones applied separately. Whereas 3 hours of treatment with either auxin or cytokinin increased SYAC1 expression (2.47±0.20 and 1.53±0.19, respectively), application of both hormones simultaneously resulted in 16.36±0.14 higher expression compared to an untreated control. This SYAC1 expression profile in roots was further validated by quantitative real-time (RT-qPCR) (FIG. 2A). To examine the spatio-temporal pattern of SYAC1 expression and its responsiveness to hormones in roots, the SYAC1 promoter was cloned and used with GUS and nuclear localized GFP reporters. The basal expression of both pSYAC1: GUS and pSYAC1:nlsGFP reporters was under the threshold of detection, however exposure to cytokinin for 6 hours enhanced reporter signal in the quiescent center (QC) and columella initials (CI) (FIG. 2B). When treated with auxin the activity of pSYAC1 in the QC and provasculature of the root apical meristem could be detected (FIG. 2B). The promoter activity of pSYAC1 was substantially enhanced by the simultaneous application of both hormones, and remarkably, a strong reporter signal was detected in the differentiation and rapid elongation zone, a pattern not observed in roots exposed to either of the hormones separately (FIG. 2B). As initial concentrations of 1 μM auxin and 10 μM cytokinin for transcriptome profiling within 3 hours were relatively high, the sensitivity of the pSYAC1 reporter line to varying concentrations of both hormones was tested. This confirmed that pSYAC1 sensitively responds to simultaneous application of both hormones, and that their supply in concentrations of 0.25 μM each is sufficient to trigger reporter expression in the root differentiation and elongation zone. These results demonstrate that the SYAC1 promoter is under the tight control of the combined and synergistic action of auxin and cytokinin. When each hormone is applied individually, SYAC1 expression is activated in cells known to exhibit either auxin or cytokinin response maxima, such as QC/columella initials or cells of the provasculature, respectively. Intriguingly, SYAC1 transcription in the root differentiation and elongation zone is fully dependent on the simultaneous enhancement of both auxin and cytokinin.

As application of cytokinin and auxin might lead to deregulation of other hormonal pathways, in particularly that of ethylene, the sensitivity of SYAC1 to this hormone was also examined. No enhancement of pSYAC1:GUS expression was detected in roots treated with either 1-aminocyclopropane-1-carboxylic acid (ACC, a precursor of ethylene biosynthesis) only or in combination with either cytokinin, auxin or both hormones together. Taken together, the expression analysis confirms SYAC1 as a novel common target of the auxin and cytokinin pathways acting in roots.

SYAC1 Expression in Planta Spatio-Temporally Correlates with Reduced Cell Growth

To explore in which growth and developmental processes SYAC1 might be involved, its expression was monitored throughout the lifespan of Arabidopsis thaliana. Strong SYAC1 expression was detected in the embryonic hypocotyl and cotyledons, but not in the embryonic root (FIG. 2D). During germination, in 2-day-old seedlings, the pSYAC1:GUS activity remained strong in cotyledons and upper part of hypocotyl, however in the lower hypocotyl its expression ceased almost completely with commencing rapid elongation growth (FIG. 2E). As growth of hypocotyl and cotyledons progressed, in 3 to 4-day-old seedlings, SYAC1 expression in these organs gradually attenuated (FIG. 2F, G). In etiolated seedlings the SYAC1 expression was concentrated in short cells at the inner (concave) side of the apical hook, whereas no signal was detected in expanded cells at outer side of the hook (FIG. 2H). Based on these data, SYAC1 function seems not be limited to plant roots and its expression pattern spatio-temporally correlates with processes involving the control of elongation growth.

SYAC1 Regulates Elongation Growth of Plant Organs

To gain insights into the developmental function of SYAC1, detailed phenotypic analysis of plants with either a lower or an enhanced activity of this gene was performed. Characterization of the available mutant alleles revealed that the T-DNA is inserted either in the middle of the 3′ untranslated region (UTR) (syac1-1, syac1-2, syac1-3) or in the middle of the second intron (syac1-4), and thus is not fully suppressing SYAC1 expression. To obtain a syac1 knock-out line, we used the CRISPR/Cas9 approach was used. In the syac1-5 the CRISPR/Cas9 cassette introduces an extra thymine at 90 bps after the ATG, which results in a STOP codon after 33 amino acids in the SYAC1 coding sequence. Additionally, to investigate the impact of increased SYAC1 activity on plant development, the genetically modified lines SYAC1-HAox, HA-SYAC1ox, SYAC1-GFPox and GFP-SYAC1ox carrying SYAC1 fused to either the -HA tag or a GFP reporter under control of the 35S promoter were generated.

Given the observed pattern of SYAC1 expression, studies were focused on growth processes involving the control of cell expansion such as apical hook development, hypocotyl elongation, and primary root growth. Specific expression at the concave side of the apical hook prompted investigation into the SYAC1 function in this developmental process. In control Arabidopsis seedlings, shortly after germination (about 15-20 h), the hypocotyl progressively bent to establish an apical hook with an angle around 180° (formation phase). This bend was stabilized during the maintenance phase and subsequently, about 60 h after germination, a progressive opening of the hook occurred (opening phase). Overexpression of SYAC1 prevented formation of the apical hook bend, severely interfering with apical hook development. In contrast, in syac1-3 and syac1-5, the formation phase occurred at a similar rate to the wild-type control, but the maintenance phase was shortened and the opening of the hook started already 35 hours after germination. Introduction of pSYAC1:gSYAC1-GFP into the syac1-3 background rescued this defect and prolonged the maintenance phase until 60 h after germination as observed in wild-type seedlings. Apical hook development is the result of tightly orchestrated differential growth along the apical-basal axis of the hypocotyl. Since the SYAC1 expression maximum occurs in the shorter, concave side of the apical hook curvature (FIG. 2H), these data suggest that local accumulation of SYAC1 restricts expansion of cells locally at the inner side of hook and thereby coordinates the timely transition of the closed apical hook to the opening phase. Disruption of this endogenous expression pattern in SYAC1ox leads to inhibition of cell expansion on both sides of the hypocotyl which prevents formation of the apical hook. Hence, SYAC1 might play an important role in the regulation of differential growth, possibly by fine tuning cell elongation. Consistent with this notion, modulation of SYAC1 activity affected growth of hypocotyls. In 4-day-old dark-grown etiolated seedlings hypocotyls were significantly longer in both syac1-3 and syac1-5 alleles, whereas SYAC1 overexpression resulted in severe reduction of hypocotyl length when compared to the wild-type control. Since hypocotyl growth in darkness is largely driven by cell elongation rather than cell proliferation, the hypocotyl growth defects observed in syac1 mutants and SYAC1ox further support the SYAC1 function in regulation of cell elongation.

Close analysis of root growth did not reveal any significant alterations in syac1-3 and syac1-5 compared to the wild-type when grown on either control or hormone supplemented media. It was therefore tested whether SYAC1 might operate in root growth adaptation to transient hormonal fluctuations. 5-day-old syac1-3 and syac1-5 seedlings revealed significantly reduced sensitivity to transient increases of auxin and cytokinin when compared to wild-type. It is therefore hypothesized that under constitutive hormonal treatment conditions other proteins might compensate for the absence of SYAC1. An in silico search for SYAC1 related genes in the Arabidopsis genome identified a family of eight highly similar (40-60%) homologous genes of which seven are located as a cluster on chromosome 1. Among these, we found that BROTHER OF SYAC1 (BSYAC1), a close homologue of SYAC1, is also synergistically regulated by auxin and cytokinin, and thus presumably partially compensates for the loss of syac1 activity. By contrast, overexpression of SYAC1 significantly reduced root length when compared to wild type. Monitoring root growth revealed that estradiol-triggered induction of SYAC1 expression triggered a steep deceleration in root growth and indicating that SYAC1 effectively feeds back onto the kinetics of root elongation. In sum, these results suggest that SYAC1 acts as a developmentally specific regulator of elongation growth, whose activity is required to coordinate specific phases of apical hook development as well as the growth of other organs, such as hypocotyls and roots.

SYAC1 Localizes to the Secretory Pathway Compartments

To explore SYAC1's cellular function, its subcellular localization in Arabidopsis root cells was compared with specific reporters for cellular compartments. In the estradiol inducible line, 5 hours after induction SYAC1-GFP is restricted to small compartments in the cell interior. Measurement of Pearson correlation coefficient revealed a high SYAC1 co-localization pattern with Golgi and trans-Golgi (TGN) compartments labeled by the anti-SEC21 (0.57±0.01) and anti-ECH (0.51±0.02) antibody, respectively. This subcellular localization was further confirmed by anti-ARF1 (0.45±0.02) and anti-SYP61 (0.49±0.02) antibodies, which label both Golgi and TGN. A significant co-localization was also observed with the prevacuolar/endosomal compartments (PVC) labeled with a mixture of anti-ARA7 and anti-RHA1 (0.52±0.02) antibodies. In contrast, almost no co-localization was observed between SYAC1 and anti-BIP2 (0.04±0.04) and anti-PIN2 (0.03±0.04) antibodies, which label ER and plasma membrane, respectively. Accordingly, the SYAC1-GFP signal in SYAC1-GFPox line exhibited strong co-localization with markers for Golgi (anti-SEC21; 0.55±0.02), TGN (anti-ECH 0.60±0.02), and for both of them together (anti-ARF1; 0.55±0.02 and anti-SYP61; 0.40±0.02) and PVC (anti-ARA7/anti-RHA1; 0.44±0.02) but almost no co-localization with markers for ER (anti-BIP2; 0.01±0.03) and the plasma membrane (anti-PIN2; 0.02±0.02). To further validate the immunocolocalization results, the GFP-SYAC1ox line was crossed with the multicolor ‘Wave’ marker set (Geldner et al., 2009) for analysis of plant endomembrane compartments. This confirmed co-localization of SYAC1 with markers for Golgi (wave 18R; 0.53±0.03 and wave 127R; 0.42±0.02), Golgi and endosomes (wave 25R; 0.69±0.03 and wave 29R; 0.35±0.03), Golgi and TGN (SYP61:SYP61-CFP; 0.45±0.02), TGN and early endosomes (wave 13R; 0.27±0.06) as well as for endosomes/recycling endosomes (wave 34R; 0.31±0.05 and wave 129R; 0.33±0.02). In agreement with immunocolocalization, SYAC1 displayed only minor co-localization with markers for ER/plasma membrane (wave 6R; 0.06±0.02), plasma membrane (wave 131R; 0.02±0.02 and wave 138R; 0.02±0.03) and vacuoles (wave 9R; 0.03±0.02). These results strongly support that SYAC1 largely resides in the Golgi, TGN, and Endosomal and PVC compartments.

SYAC1 is a Novel Component of the ECHIDNA/Yip Complex

To further assess SYAC1's molecular function, its molecular interactors were identified using a tandem affinity purification assay with SYAC1 as bait. Proteins including the integral membrane YIP1 family protein (YIP5b), β-ketoacyl reductase 1 (KCR1), an ubiquitin receptor protein (DSK2), and prohibitin 4 (PHB4) were recovered by this approach. As YIP5b is a member of the YIP (for YPT/RAB GTPase Interacting Protein) family in Arabidopsis thaliana that forms a TGN-localized complex with YIP4a and YIP4b homologues and Echidna (ECH) integral membrane protein, they were included them in subsequent detailed interaction studies. A Yeast two-hybrid assay (Y2H) revealed a strong interaction between SYAC1 and all three YIP family members. Moreover, SYAC1 interacted well with ECH, but only weakly with KCR1 and not at all with the DSK2 and PHB4 proteins. The Y2H results were further validated in planta using a bimolecular fluorescence complementation (BiFC) assay. SYAC1 tagged with the C-terminus of EYFP, and YIP5b, YIP4a, YIP4b, ECH, KCR1, DSK2 and PHB4 tagged with the N-terminus of EYFP, were transiently expressed in an Arabidopsis root suspension culture. Yellow fluorescence was detected in protoplasts overexpressing SYAC1 in combination with YIP5b, YIP4a, YIP4b and ECH, indicating the close physical proximity of these proteins in vivo. By contrast, no EYFP reconstitution was detected in cells overexpressing SYAC1 with KCR1, DSK and PHB4, respectively, in agreement with the result of the Y2H assay. Finally, the interaction between SYAC1 and YIP4a and between SYAC1 and ECH was also confirmed by a co-immunoprecipitation (Co-IP) assay. Results from tandem affinity purification, BiFC and Co-IP assays revealed SYAC1 interaction with YIP5b, YIP4a, YIP4b and ECH protein, and indicate its function in the protein complex involved in maintaining the functionality of the secretory pathway.

SYAC1 Determines Secretory Pathway Activity

SYAC1 localization in Golgi/TGN/Endosomal/PVC compartments and the interaction with ECH/YIPs pointed towards a potential function in the secretory pathway. The secretory pathway is of vital importance for all eukaryotic cells, since it manufactures, stores and distributes macromolecules, lipids and proteins as cargo to intracellular and extracellular locations. To assess the involvement of SYAC1 in the regulation of secretion, transient expression assays were performed in Arabidopsis mesophyll protoplasts and the impact assessed of SYAC1-HAox or HA-SYAC1ox on the secretory index of the α-Amylase (Amy) reporter—a protein that without any intrinsic sorting signal is secreted extracellularly and can be detected by its endogenous enzymatic activity. The secretion index was determined by quantifying the ratio of the α-Amylase activity in the medium and in the cells. Expression of the SYAC1 protein decreased the secretion index from 0.70±0.04 in control sample to 0.55±0.02 (SYAC1-HAox) and 0.45±0.01 (HA-SYAC1ox), which suggests a function of SYAC1 as a negative regulator of the anterograde secretory route to the cell surface. Because of SYAC1's co-localization with markers for PVC compartments, SYAC1's involvement in transport to the vacuoles was investigated. For that, an α-Amylase with a vacuolar sorting signal (Amy-Spo) was co-transfected with either SYAC1-HA or HA-SYAC1 encoding plasmids. The secretion index of α-Amylase (Amy-Spo) was increased from 0.07±0.01 in the control sample to 0.29±0.01 (SYAC1-HAox) and 0.28±0.03 (HA-SYAC1ox), which suggests that SYAC1 impairs transport to vacuoles leading to more α-Amylase secretion out of the cells. SYAC1's effect on α-Amylase containing an ER retention signal (Amy-HDEL), which redirects the protein back to the ER was tested. Co-transfection of SYAC1 significantly decreased the secretion index in protoplasts with leaky retention of α-Amylase from 0.34±0.01 in the control sample to 0.24±0.01 (SYAC1-HAox) and 0.26±0.04 (HA-SYAC1ox). Altogether these results support the conclusion that SYAC1 modulates the activity of the secretory pathway, and coordinates cargo trafficking towards the extracellular space and vacuoles.

The Effect of SYAC1 Expression on Plant Cell Wall Composition

In plants, new cell wall components such as pectins and hemicellulose are proposed to be delivered to the extracellular matrix via the secretory pathway (reviewed in Wolf and Greiner, 2012, Protoplasma 249, 169-175). SYAC1 reduction of α-Amylase secretion, along with its Golgi/TGN/Endosomal localization and interaction with YIPs and Echidna proteins suggested a role for SYAC1 in the control of soluble cell wall polysaccharides (pectin and hemicellulose) secretion. Investigating the seed coat epidermis, whose TGN is highly specialized for pectic mucilage secretion using ruthenium red staining assay revealed that mucilage release from mature seeds was greatly reduced in SYAC1-GFPox seeds, relative to wild-type (FIG. 7), which is in line with the anticipated function of SYAC1 as an inhibitor of polysaccharide secretion and with the previously described ech-1 and yip4ayip4b mutants.

Delivery of new cell wall components during pollen tube growth is a particularly active process. The effects of SYAC1 expression on pectin secretion in tobacco pollen tubes was studied and it was observed that SYAC1 severely affected accumulation of pectin at the pollen tip, supporting its role as a factor modulating the activity of the secretory pathway (FIG. 10). As in Arabidopsis root cells, the localization of plasma membrane proteins such as PIN1 and PIN2 was not affected, suggesting that SYAC1 might preferentially regulate specific branches of the secretory pathway. Taken together, these data support SYAC1 function in modulation of the cell wall matrix polysaccharides delivery.

To assess the impact of the modulated activity of SYAC1 on cell wall composition, hypocotyls of etiolated seedlings were inspected using Fourier transform-infrared (FT-IR) microspectroscopy. FT-IR analysis revealed that enhanced SYAC1 activity in plant cells substantially alters the composition of cell walls, which is manifested by a significantly reduced proportion of carbohydrates (FIG. 3). To further dissect the qualitative changes in the cell wall, analyses of pectin content and xyloglucans, two major components of cell walls, in hypocotyls of seedlings with modified SYAC1 expression were performed. A quantitative analysis of galacturonic acid, a key structural component of pectin, did not reveal any significant changes in hypocotyls of the syac1 loss of function mutant when compared to the control, consistent with the relatively modest defects on syac1 hypocotyl growth. By contrast, the amount of galacturonic acid extracted from hypocotyls of seedlings with enhanced expression of SYAC1 was significantly reduced when compared to control (FIG. 5C). The pectic polysaccharides are secreted in a methylesterified form. In correlation with a reduced amount of pectin in the cell wall, methanol content was decreased in hypocotyls of seedlings with enhanced SYAC1 activity. Unlike pectin, no changes in the amount of fucose, galactose and xylose which are indicators of xyloglucan content in the cell wall could be detected in seedlings with either loss or enhanced activity of SYAC1. Altogether these results suggest that SYAC1 interferes with a specific branch of the secretory pathway mediating pectin delivery to the cell wall.

Secretion defects which lead to alterations in cell wall composition might ultimately result in changes in cell wall physical properties, and on plant wall stiffness in particular. Analyses of etiolated hypocotyls using atomic force microscopy (AFM) revealed a significantly reduced apparent Young modulus on the extracellular matrix upon enhanced SYAC1 activity when compared to control (FIG. 4). Altogether, these results support the conclusion that SYAC1 acts as a regulator of the TGN-mediated secretion of cell wall components such as pectins and ultimately affects the composition and physical properties of cell walls.

The results presented in FIG. 4, which compares a control plant: Arabidopsis thaliana (L.) Heynh ecopyte Columbia and with a genetically modified plant according to the invention: SYAC1-GFPox, confirm a difference in stiffness between the cell walls on control plants and the cell walls of SYAC1 overexpressor (SYAC1-GFPox) plants. This reduced stiffness of the cell walls in the genetically modified plants increases the efficiency of cell wall-degrading enzymes and of the extraction of polysaccharides.

SYAC1 as a Spatio-Temporal Modulator of YIP/ECH Complex Function

SYAC1 was shown to interact with YIPs and ECH, components of the protein complex required for the proper operation of the secretory pathway. Intriguingly, compromised functionality of the YIP/ECH complex leads to cellular and developmental defects reminiscent of these caused by enhanced activity of SYAC1. Similarly to SYAC1ox, deficiency in the secretion of pectins, as well as root, hypocotyl and apical hook development defects have been observed in yip4a, yip4b and ech loss of function mutants. To explore SYAC1 function as a potential attenuator of YIP/ECH complex activity, tests were carried out to determine whether relief of the SYAC1 mediated inhibition of the secretory pathway might alleviate growth defects caused by defects of the YIP/ECH complex. To test this hypothesis, the syac1-3 allele was introduced into the yip4a, yip4b mutant background, and the resulting combined genotype was phenotyped. Importantly, the syac1-3 yip4a yip4b triple mutant displayed significantly improved growth of hypocotyls and shoot organs when compared to the yip4a, yip4b double mutant, indicating that SYAC1 might indeed act as a negative regulator of the YIPs/ECH complex. Based on these observations it is hypothesized that whereas constitutively expressed YIPs and ECH act as generic factors required for the secretion of cell wall components to maintain cell expansion, the spatio-temporally controlled expression pattern of SYAC1 may allow it to act as a developmentally specific regulator of the YIP/ECH complex to fine tune secretory pathway activity and thereby plant organ growth.

SYAC1 Expression Results in an Increase in Hemicellulose Expression in Plant Cell Walls

In order to demonstrate that SYAC1 expression results in an increase in hemicellulose expression in plant cell walls, xyloglucan levels were detected using a labelled antibody. FIG. 5A and FIG. 5B demonstrate, using immunolacalization of xyloglucan with an LM 15 antibody, an increase and change in xyloglucan localization in SYAC1-GFPox line. The immunolabeling was performed as described above.

SYAC1 Expression Results in Reduction in Galacturonic Acid in Plant Cell Walls

FIG. 5C demonstrates that when the expression of SYAC1 is increased, the amount of galacturonic acid in the plant cell wall decreases. Analysis of the cell wall was performed on 4 days old dark grown hypocotyls as described above.

SYAC1 Expression Affects the α-Amylase Secretion Index

α-Amylase assays and calculations of the secretion index were performed as described by Früholz and Pimpl (2017) in Plant Protein Secretion, (Humana Press, New York, N.Y.), pp. 171-182. α-Amylase expression constructs were kindly provided by P. Pimpl and transfections were performed in Arabidopsis mesophyll protoplasts. α-Amylase activity was measured with the Ceralpha kit from Megazyme. The reaction was performed in a microtiter plate at 37° C. with 30 μL of extract and 30 μL of substrate. The reaction was stopped by the addition of 150 μL of stop buffer. The absorbance was measured at a wavelength of 405 nm. Experiment was performed three times with four replicates.

These results shown in FIG. 6 indicate that SYAC1 modulates activity of a secretory pathway, and coordinates trafficking of cargos towards extracellular space in plant cells. The changes observed in the cell wall modification in SYAC1ox lines are caused by regulation of secretory pathway. This mechanism clearly distinguishes SYAC1 from other genes known to regulate cell wall composition. Results shown in FIGS. 13A-C indicate similar regulation of secretory pathway also by SYAC1 paralogues.

SYAC1 Expression Inhibits Seed Coat Mucilage Secretion

In order to demonstrate that SYAC1 expression inhibits seed coat mucilage secretion, mature seeds of Arabidopsis thaliana (L.) Heynh ecopyte Columbia and SYAC1-GFPox were incubated in 0.01% (w/v) aqueous solution of Ruthenium red. Seeds were mounted in water and viewed using a DIC Olympus BX53 microscope.

The results in FIG. 7 show that mucilage release from mature seeds is significantly reduced in SYAC1-GFPox seeds, relative to Columbia control cells. The result confirms that SYAC1 plays a role in the regulation of cell wall components secretion.

SYAC1 Expression Improves Pathogen Defence

To examine whether SYAC1 is useful in host-pathogen interaction and defence the sensitivity of plants with altered SYAC1 expression to the pathogen Plasmodiophora brassicae was studied. The analysis of root and shoot phenotypes after inoculation with the plant pathogenic protist was done at three different spore concentrations (10⁶, 10⁵ and 10⁴ spores mL-1). While a high inoculation pressure should identify tolerant plants, low spore concentrations will reveal oversensitive phenotypes. To characterize disease progression 5 categories was used (Siemens et al., 2002, Journal of Phytopathology 150, 592-605): zero denotes no infection, 1 almost no infection, 2 and 3 intermediate infection phenotypes and 4 a root completely transformed into a clubroot. Both syac1-3 and syac1-5 mutant alleles exhibited increased tolerance to the pathogen when compared to the wild-type control, unlike plants overexpressing SYAC1 which exhibited oversensitivity to pathogen infection (FIGS. 11 and 12). Both root and shoot phenotypes of infected plants are in line with these observations. In both mutant plant lines more roots were classified into lower disease classes at high and medium spore concentrations than in wild-type while at the low spore concentration no differences were observed (FIG. 11). On the contrary, all SYAC1 overexpressors showed more roots in class 4 when compared to the wild-type at the lowest spore concentration indicating their oversensitivity to the pathogen. Rosette development followed the same pattern (FIG. 12). Altogether these results indicate that SYAC1-mediated control of cell wall composition in roots is of crucial importance for root-pathogen interaction and the suppression of SYAC1 expression in roots could be the result of evolutionary selection to restrain invasions of pathogens through the root system.

The results demonstrated that by reducing the expression of SYAC1 in a plant, such as a a Brassica species, susceptibility to some pathogenic organisms, such as fungi, can be reduced. In particular the susceptibility of Brassica species to diseases caused by Plasmodiophora brassicae, such as club root disease can be reduced. A reduction in susceptibility to a pathogenic organism may result in a reduced ability of the organism to colonise the plant.

The data presented herein demonstrates that where plants were infected with 10⁵ spore concentrations of P. brassicae, almost 60% of syac1 mutant lines showed reduced susceptibility to the pathogen in comparison to only 30% of wild-type plants. With increased spore concentration (10⁶) still almost 40% of mutant lines maintained resistance, compared to only 15% in wild-type plants. In addition to clubroot resistance, earlier flowering was also observed in syac1 mutant lines which could potentially be of interest for reducing time needed to finish the life cycle. On the contrary, all SYAC1 overexpressors showed increased sensitivity to pathogen compared to the wild-type already at the lowest spore concentration.

SYAC1 Expression Renders Plant Cells More Tolerant to Copper

FIG. 8 shows the effect of excess copper on the primary root growth of seedlings grown on agar. Plants were grown for 5 days on normal AM+ media and then transferred to media containing 50 μM CuSO₄. CuSO₄ is shown to trigger swelling of cells at the root tip of wild type control plants, but not SYAC1-GFPox plants. These results demonstrate that SYAC1 expression renders plant cells more tolerant to elevated copper.

FIG. 9 further supports the ability of SYAC1 expression to render plants suitable for cultivation on contaminated soil. In particular, the results demonstrate that SYAC1 expression significantly reduces the amount of Cu or Fe taken up by plant roots. Wild type Col-0 and the SYAC1 overexpressor lines (HA-SYAC1ox) were grown for 1 week in standard MS media on agar plates. Fresh roots were collected and rinsed in deionized water during the sampling and stored in Falcon tubes. To remove the copper in the cell apoplast, fresh roots samples were immediately washed once in 40 ml 1 mM HCl solution (shaking end over end) for 3 mins. First washing solution was removed and samples were additionally washed in 0.01M HCl for 5 mins. Finally, root samples were rinsed in 40 ml deionized H₂O (Chaignon and Hinsinger, J. Environ. Qual., 2003). Samples were dried overnight in Falcon tubes at 65° C. in oven. For analysis of metal content, sample digestion has been performed at 150° C. for 4 h in a mixture of 65% HNO₃ and 30% H₂O₂ in a ratio of 5:1 (v/v). Copper and iron were measured by detecting two isotopes, Cu-63 and Cu-65 or Fe-54 and Fe-57 respectively in a Perkin Elmer Elan DRCe 9000 (Rosenkranz et al., Waste Manag., 2018).

SYAC1 Expression in Tobacco Pollens Tubes Alters Pectin Secretion

Mature pollen was collected from four to six tobacco (Nicotiana tabacum) flowers of 8-week-old plants. Pollen was resuspended in growth medium, filtered onto cellulose acetate filters, and transferred to Whatman paper moistened with growth medium. Within 5 to 10 min of harvesting, pollen was transformed by bombardment with plasmidcoated 1-mm gold particles with a helium-driven particle accelerator (PDS-1000/He; Bio-Rad) using 1350 p.s.i. rupture discs and a vacuum of 28 inches of mercury. Gold particles (1.25 mg) were coated with 3 to 7 mg of plasmid DNA. After bombardment, pollen was resuspended in growth medium and grown for 5 to 14 h in small droplets of media directly on microscope slides. Digital images were taken under the light microscope (Olympus BX51) within 5 to 15 min after addition of the dye. Pollen tubes transformed with eYFP (enhanced Yellow fluorescent protein) were taken as control to pollen tubes transformed with SYAC1 tagged with mCherry protein)

The data presented in FIGS. 10A and 10B clearly show that the expression of SYAC1 in the pollen tubes results in a change in the normal pattern of pectin secretion, when compared to a control pollen tube which does not express SYAC1.

### CONCLUSION

The results presented here demonstrate that SYAC1 expression results in a reduction of α-amylase secretion, along with the observation that it localizes in the golgi/TGN/endosomes and interacts with YIPs and Echidna proteins, this demonstrates a role for SYAC1 in the control of soluble cell wall polysaccharides (pectin and hemicellulose) secretion. Investigating the seed coat epidermis, in which the TGN is highly specialized for pectic mucilage secretion using ruthenium red staining assay revealed that mucilage release from mature seeds was greatly reduced in SYAC1-GFPox seeds, relative to Columbia (FIG. 7) which is in line with a function of SYAC1 as a regulator of polysaccharide secretion. Hemicellulose components of cell wall, monitored using LM15 an anti-xyloglucan antibody, were enriched around QC (quiescent centre) in Columbia control roots. In the SYAC1-GFPox line, enhanced staining of root epidermal, cortex and endodermal was detected. Taken together, this data supports a role for SYAC1 in the modulation of cell wall matrix polysaccharides delivery, including both hemicelluloses and pectins. The defects in the secretion of cell wall components leads to alterations in the cell wall structure and its physical properties. To assess whether SYAC1 triggered changes in delivery of cell wall components affect the composition and physical properties of cell walls, hypocotyls of etiolated seedlings were inspected using Fourier transform-infrared (FT-IR) microspectroscopy and atomic force microscopy (AFM). FT-IR analysis revealed that enhanced SYAC1 activity in plant cells substantially alters the composition of the cell walls, which is manifested by a significantly reduced proportion of carbohydrates (FIG. 3). Accordingly, an increase in SYAC1 activity has a direct impact on the physical properties of cell walls and reduces their stiffness (FIG. 4). Thus, these results indicate that SYAC1 is an essential regulator of the TGN-mediated secretion of cell wall components such as pectins and xyloglucan and ultimately affects the composition and physical properties of cell walls.

The efficient conversion of lignocellulosic biomass into fuel ethanol has become a world priority for producing environmentally friendly renewable energy at a reasonable price for the transportation sector. The main source of lignocellulose is plant secondary cell walls, this is the thick, strengthening layer of the cell wall that is laid down inside the primary wall after cell elongation has terminated. Approximately 75% of lignocellulose is comprised of polysaccharides, which can potentially be converted into monosaccharides for fermentation. The main constituents of plant secondary cell walls are cellulose, hemicellulose and lignin, and these are present in varying proportions in different feedstocks. The cellulose fibres are embedded in a matrix of hemicellulose and, in primary cell walls, pectin, which appear to play a dual role of strengthening and increasing extensibility of the wall to enable cell enlargement. In secondary cell walls, the polysaccharide network is impregnated and coated by lignin, which provides rigidity and strength. Lignin strengthens the cell wall, increasing its hydrophobicity and posing a formidable barrier to cell wall-degrading enzymes. Plant cell wall softening caused by SYAC1 overexpression, and demonstrated herein, leads to a decrease of hydrophobicity and opens the way for degrading enzymes. Similarly, an increase in hemicellulose content provides new material for sugar extraction. Thus plants or plant cells with increased SYAC1 levels are a good source of readily accessible polysaccharides for use as biomass in energy production.

1. A genetically modified plant or plant cell transformed with an isolated nucleic acid, wherein the isolated nucleic acid encodes a SYAC1 protein or a protein related to the SYAC1 protein or a protein with at least about 80%, 85%, 90%, 95%, 98%, 99% or more sequence identity with a SYAC1 protein.

2. A genetically modified plant or plant cell transformed with an isolated nucleic acid, wherein the isolated nucleic acid comprises or consists of sequence selected from the group consisting of Seq ID no: 1, Seq ID no: 2, Seq ID no: 3, Seq ID no: 4, Seq ID no: 5, Seq ID no: 6, Seq ID no: 7 or Seq ID no: 8, or a sequence that has at least 80%, 85%, 90%, 95%, 98%, 99% or more sequence identity with one of Seq ID no: 1, Seq ID no: 2, Seq ID no: 3, Seq ID no: 4, Seq ID no: 5, Seq ID no: 6, Seq ID no: 7 or Seq ID no: 8.

3. The genetically modified plant or plant cell of claim 1 wherein the isolated nucleic acid encodes a polypeptide having the sequence of Seq ID no: 9, Seq ID no: 10, Seq ID no: 11, Seq ID no: 12, Seq ID no: 13, Seq ID no: 14, Seq ID no: 15 or Seq ID no: 16, or a sequence that has at least about 80%, 85%, 90%, 95%, 98%, 99% or more sequence identity with one of Seq ID no: 9, Seq ID no: 10, Seq ID no: 11, Seq ID no: 12, Seq ID no: 13, Seq ID no: 14, Seq ID no: 15 or Seq ID no: 16.

4. The genetically modified plant or plant cell of claim 1 wherein the expression of the isolated nucleic acid is under the control of the SYAC1 promoter of Seq ID no: 17, or a sequence that has at least about 80%, 85%, 90%, 95%, 98%, 99% or more sequence identity with Seq ID no: 17, or a functional fragment thereof.

5. The genetically modified plant or plant cell of claim 1 which expresses an elevated level of SYAC1 protein or a protein related to the SYAC1 protein, compared to a wild type plant or plant cell.

6. (canceled)

7. The genetically modified plant or plant cell of claim 1 which has one or more of the characteristics selected from the group consisting of a) an increased level of hemicellulose in the cell walls compared to a wild type plant of plant cell; b) less rigid cell walls compared to a wild type plant of plant cell; c) an increased tolerance to growth on contaminated soil compared to a wild type plant or plant cell; and d) altered susceptibility to one or more microbes.

8. The genetically modified plant or plant cell of claim 1 wherein the plant or plant cell is more resistant to disease caused by Plasmodiophora brassicae than a wild type plant or plant cell.

9. The genetically modified plant or plant cell of claim 8 wherein the plant or plant cell has a reduced level of, or no, SYAC1 protein or a protein related to the SYAC1 protein, compared to a wild type plant or cell.

10. The genetically modified plant or plant cell of claim 1 wherein the plant or plant cell has an increased susceptibility to a symbiont or to other beneficial microorganisms.

11. The genetically modified plant or plant cell of claim 10 wherein the plant or plant cell has an elevated level of SYAC1 protein or a protein related to the SYAC1 protein, compared to a wild type plant.

12. The genetically modified plant or plant cell of claim 1 wherein the plant is a monocotyledonous plant or a dicotyledonous plant.

13. The genetically modified plant or plant cell of claim 1 where the spatial and/or temporal expression of the SYAC1 protein or a protein related to the SYAC1 protein, is controlled by a hormone, such as auxin and/or a cytokinin, and/or the copper concentration.

14. (canceled)

15. (canceled)

16. A seed produced by a genetically modified plant or plant cell of claim 1.

17.-20. (canceled)

21. A promoter comprising or consisting of the sequence of SEQ ID NO: 17, or a sequence that has at least about 80%, 85%, 90%, 95%, 98%, 99% or more sequence identity with SEQ ID NO: 17, or a functional fragment thereof.

22.-28. (canceled)
